(19) United States
(12) Patent Application Publication
(10) Pub. No.: US 2022/0313843 A1
(43) Pub. Date: Oct. 6, 2022

(54) NUCLEIC ACID SEQUENCE FOR REGULATION OF TRANSGENE EXPRESSION

(71) Applicants: ASSOCIATION INSTITUT DE MYOLOGIE, PARIS (FR); INSERM (INSTITUT NATIONAL DE LA SANTÉ ET DE LA RECHERCHE MÉDICALE), PARIS (FR); SORBONNE UNIVERSITE, PARIS (FR)

(72) Inventors: DENIS FURLING, JOINVILLE-LE-PONT (FR); LUDOVIC ARANDEL, SAINT MAUR DES FOSSES (FR); MAGDALENA MATLOKA, PARIS (FR); ALAIN SUREAU, BRUNOY (FR); MICHEL NEY, ANTONY (FR)

(21) Appl. No.: 17/642,244

(22) PCT Filed: Sep. 11, 2020
(86) PCT No.: PCT/EP2020/075574

§ 371 Date: Mar. 11, 2022

(30) Foreign Application Priority Data
Sep. 12, 2019 (EP) 19306099.3

(51) Int. Cl. A61K 48/00; C12N 15/63; C12N 15/86

(52) U.S. Cl. CPC A61K 48/0066; A61K 48/0075; C12N 15/635; C12N 15/86

(57) ABSTRACT

The present invention relates to the field of gene therapy. In particular, the invention relates to a nucleic acid molecule comprising a nucleic acid sequence able to regulate the expression of a transgene of interest, to a vector or a cell comprising said nucleic acid molecule, and uses thereof.

### FIELD OF THE INVENTION

The present invention relates to the field of gene therapy. In particular, the invention relates to a nucleic acid molecule comprising a nucleic acid sequence able to regulate the expression of a transgene of interest, to a vector or a cell comprising said nucleic acid molecule, and uses thereof.

### BACKGROUND OF THE INVENTION

Gene therapy is a strategy based on the transfer of a therapeutic transgene into a cell or a host organism. Its concept, first considered in the context of genetic diseases, was quickly expanded to the treatment of a large number of other pathologies, such as cancers, infectious diseases, or cardiovascular diseases. Examples of pathologies treated by gene therapy include diseases linked to the alteration of the function of a protein regulating alternative splicing, such as myotonic dystrophy. Myotonic dystrophy (DM) is due to the loss-of-function of the MBNL protein, which results in mis-splicing events leading to symptoms of DM. Myotonic dystrophy type 1 (DM1), one of the most common neuromuscular disorders in adult, is an inherited autosomal dominant disease caused by an unstable CTG expansion located in the 3′ untranslated region (UTR) of the dystrophia myotonica protein kinase (DMPK) gene (Brook et al. 1992). The expanded CTG repeats are transcribed into CUG repeats that accumulate as aggregates in “nuclear foci”. MBNL protein, which is involved in the regulation of alternative splicing, binds to expanded CUG repeats with high affinity, and colocalizes with nuclear foci of CUGexp-RNA in DM1 muscle cells (Miller et al. 2000; Fardaei et al. 2001). Sequestration of MBNL in these nuclear foci leads to its loss-of-function, and consequently to alternative splicing misregulation of several pre-mRNAs targeted by MBNL.

In the context of DM1, gene therapy vectors were proposed to express therapeutic transgene aiming to target either expanded repeats, DMPK transcripts or DMPK gene and/or to restore functional activity of MBNL protein. For example, overexpression of functional and full length MBNL1 (isoform 41) in the skeletal muscles of DM1 mice was shown to correct splicing defects and abolish myotonia, hallmarks of DM1 disease (Kanadia et al. 2006). Antisense oligonucleotide approaches that interfere with CUGexp-RNAs to release MBNL1 from the foci have also been proposed for reversing splicing misregulations and myotonia in a DM1 mouse model. Furthermore, in WO2015/158365, it was proposed to express a non-functional variant of MBNL protein having reduced or even no splicing activity, to compete, displace, and replace thereon endogenous MBNL protein(s) within CUGexp-foci to avoid the negative consequences of their sequestration.

However, it may be desirable to improve these gene therapy approaches by controlling the expression of the therapeutic transgene. Indeed, excessive expression of the therapeutic transgene may have unwanted effect, in particular in healthy tissues or cells wherein the expression of the therapeutic transgene is not needed. Thus, a new system that can offer tight control of transgene expression would be advantageous for improving gene therapy, such as therapy of myotonic dystrophy.

### SUMMARY OF THE INVENTION

The purpose of the invention is to provide a system that regulates the expression of a transgene of interest into a cell or a host organism, depending on the activity of a protein regulating alternative splicing within the host cell or tissue.

An aspect of this invention relates to a chimeric nucleic acid molecule comprising:
a first nucleic acid sequence, the primary transcript of which comprises an exon, said primary transcript being subject to splicing by a protein regulating alternative splicing; and
a second nucleic acid sequence comprising a transgene of interest encoding a product of interest;
   wherein the protein regulating alternative splicing enhances the inclusion of said exon in the mature transcript of the chimeric nucleic acid molecule, and
   wherein said exon is designed to inhibit expression of a functional product of interest, when included into the mature transcript.

In a particular embodiment, the first nucleic acid sequence is located upstream of the second nucleic acid sequence. In a particular embodiment, the inclusion of the exon in the mature transcript of the chimeric nucleic acid molecule leads to the occurrence of a premature STOP codon in said mature transcript. In a particular embodiment, the exon of the first sequence includes a STOP codon. In a particular embodiment, the exon of the first sequence is flanked by two introns. In particular, said exon may be exon 22 of the ATP2A1 gene, exon 70 of the RYR1 gene, exon 8 of the CAPZB gene, exon 11 of the INSR gene, exon 13 of the CAM2B gene, exon 17 of the ITGB gene, exon 11 of the BIN1 gene, exon 2 or 3 of the TAU gene or exon 78 of the DMD gene, in particular the exon 22 of the ATP2A1 gene. In a particular embodiment, said exon 22 of the ATP2A1 gene is flanked by intron 21 and intron 22 of the ATP2A1 gene.

In a particular embodiment, the transgene of interest, as recited above, encodes the protein regulating alternative splicing, or a variant thereof. In particular, the protein regulating alternative splicing can be a MBNL protein, in particular a MBNL1, MBNL2, or MBNL3 protein, preferably a MBNL1 protein, or a variant thereof. In a particular embodiment, the transgene of interest encodes a modified MBNL protein having an YGCY binding property and having a reduced splicing activity as compared to wild-type MBNL protein. More particularly, said modified MBNL protein may be able to bind CUG repeats. In a particular embodiment, said modified MBNL protein is lacking the C-terminal domain of the wild-type MBNL protein. More particularly, the modified MBNL protein may be derived from the MBNL1 protein and may lack the amino acids corresponding to exons 5 to 10 of the MBNL1 mRNA, the modified MBNL polypeptide having in particular the sequence shown in SEQ ID NO: 5 or being a functional YGCY-binding variant thereof. In a further particular embodiment, the modified MBNL protein has a splicing activity reduced by at least 50% as compared to the wild-type MBNL protein.

Another aspect disclosed herein is an expression cassette comprising the nucleic acid molecule of the invention, operably linked to regulatory sequences.

The present invention also relates to a vector comprising the nucleic acid molecule or the expression cassette of the invention. In a particular embodiment, said vector is a plasmid or a viral vector.

It is also described herein an isolated cell transformed with the nucleic acid molecule, the expression cassette or the vector of the invention.

Another aspect disclosed herein is the use of the nucleic acid molecule, the expression cassette, the vector or the isolated cell of the invention, as a medicament. In a particular embodiment, the nucleic acid molecule, the expression cassette, the vector or the isolated cell of the invention are used in the treatment of a disease or disorder that can benefit from the expression of the transgene of interest. In a particular embodiment, the disease or disorder is linked to a dysfunction of a protein regulating alternative splicing, such as a disease or disorder linked to a sequestration of MBNL, in particular for the treatment of a myotonic dystrophy, in particular for the treatment of DM1 or DM2.

It is also described herein the use of the nucleic acid molecule, the expression cassette, the vector or the isolated cell of the invention, in a method for controlling the expression of a functional product of interest, depending on the activity of the protein regulating alternative splicing.

### LEGENDS TO THE FIGURES

FIG. 1. Splice-sensor-GFP regulation. A) Schematic representation of the ATP2A1 splice-sensor sequence fused to a GFP sequence. Two codon stops are located within the exon 22. B) Schematic representation of exogenous MBNL1 expression in stably transfected HEK293T cells using an inducible Tet-on system. Addition of the tetracycline analog, doxycycline (dox), de-represses transcriptional inhibition by binding to the Tet-Repressor allowing the expression of MBNL1. Immuno-blot showing of Dox-treated cells shows exogenous MBNL1 protein expression. C) RT-PCR performed on splice-sensor-exon 22 mRNA extracted from splice-sensor-GFP transfected Hek293T cells treated or not with dox. The splice-sensor-exon 22 is excluded when the level of MBNL1 is low (−dox condition). In contrast the splice-sensor-exon 22 is included when the level of MBNL1 is high (+dox condition). D) Western blot showed that the GFP protein expression is under the control of alternative splicing of splice-sensor-ATP2A1 exon 22. Indeed, low level of MBNL1 protein leads to splice-sensor-exon 22 skipping and GFP protein expression whereas high level of MBNL1 causes splice-sensor-exon 22 inclusion and translation inhibition due to the presence of stop codons resulting in lack of GFP expression.

FIG. 2. Splice-sensor-V5-MBNLΔ regulation. A) Schematic representation of splice-sensor-MBNLΔ chimeric construct. A V5 tag was inserted between the splice-sensor-ATP2A1 exon 22 and MBNLΔ sequences. B) RT-PCR performed on splice-sensor exon 22 extracted from splice-sensor-V5-MBNLΔ transfected Hek293T cells with low (−DOX) and high (+DOX) level of MBNL1 expression. Splice-sensor-exon 22 is included when the level of MBNL1 is high (+dox condition). C) Western blot analysis showed V5-MBNLΔ protein expression in splice-sensor-V5-MBNLΔ transfected Hek293T cells expressing low level of MBNL1 protein (−dox). In contrast, no expression of V5-MBNLΔ protein was detected in splice-sensor-V5 transfected Hek293T cells expressing high level of MBNL1 (+dox).

FIG. 3. Splice-sensor controls MBNLΔ transgene expression in wild-type mice. A) Wild type (WT) mice were injected intramuscularly with AAV9 vectors expressing either V5-MBNLΔ or splice-sensor-ATP2A1 exon 22-V5-MBNLΔ constructs. Two doses of AAV9 vectors were used for each condition and mice were sacrificed five weeks later. B) RT-PCR showed that V5 MBNLΔ mRNAs were expressed from both constructs in both conditions. C) Western blot analysis showed that V5-MBNLΔ protein is only detected in AAV9-V5-MBNLΔ treated muscles. No expression was detected in AAV9-splice-sensor-V5-MBNLΔ treated muscles from WT mice at both conditions.

FIG. 4. Splice-sensor allows MBNLΔ transgene expression in DM1 mice. A) DM1 (HSA-LR) mice were injected intramuscularly with AAV9 vectors expressing either V5 MBNLΔ or splice-sensor-ATP2A1 exon 22-V5-MBNLΔ constructs. Mice were sacrificed five weeks later. B) RT-PCR showed that V5 MBNLΔ mRNAs were expressed from both constructs. Also splicing misregulation found in skeletal muscles of DM1 mice when compared to WT mice were corrected to similar extent by either V5 MBNLΔ or splice-sensor-V5-MBNLΔ constructs. C) Western blot analysis showed that V5-MBNLΔ protein is detected in DM1 muscles injected either with AAV9-V5-MBNLΔ or AAV9-splice-sensor-V5-MBNLΔ. Endogenous MBNL1 expression was used as internal control.

FIG. 5. Splice-sensor-dCas9 regulation. A) Schematic representation of the ATP2A1 splice-sensor sequence fused to a dCas9-eGFP sequence. B) RT-PCR performed on splice-sensor exon 22 mRNA extracted from splice-sensor-dCas9-eGFP transfected Hek293T cells expressing either low (−dox) or high (+dox) level of MBNL1. Splice-sensor that contains two codon stops within exon 22, is included only when the level of MBNL1 is high (+dox). C) Western blot analysis showed dCas9-eGFP protein expression in splice-sensor-dCas9-eGFP transfected Hek293T cells expressing low level of MBNL1 protein (−dox). dCas9-eGFP protein expression was reduced by 65% when the level of MBNL1 (+dox) is high in splice-sensor-dCas9-eGFP transfected Hek293T cells.

FIG. 6. Embedded-Splice-sensor-V5-MBNLΔ regulation. A) Schematic representation of the splice-sensor ATP2A1 sequence embedded into the MBNLΔ sequence. Embedded-Splice-Sensor was inserted either between MBNLΔ exon 2 and exon 3 or between MBNLΔ exon 3 and exon 4. B) RT-PCR performed on embedded-splice-sensor mRNA extracted from embedded-splice-sensor-V5-MBNLΔ transfected Hek293T cells expressing either low (−dox) or high (+dox) level of MBNL1. Embedded-splice-sensor that contains two codon stops within exon 22 is included only when the level of MBNL1 is high (+dox). C) Western blot analysis showed V5-MBNLΔ protein expression in embedded-splice-sensor-V5-MBNLΔ transfected Hek293T cells expressing low level of MBNL1 protein (−dox). When the level of MBNL1 (+dox) is high, V5-MBNLΔ protein expression was reduced by 60% in transfected Hek293T cells, when the embedded-splice-sensor is embedded between exon 2 and exon 3. When the level of MBNL1 (+dox) is high, V5-MBNLΔ protein expression was reduced by 70% in transfected Hek293T cells, when the embedded-splice-sensor is embedded between exon 3 and exon 4.

FIG. 7. 3′-Synthetic-Splice-sensor-V5-MBNLΔ regulation. A) Schematic representation of the 3′-synthetic-splice-sensor ATP2A1 sequence inserted between STOP codon and polyA of the V5-MBNLΔ sequence. The exon 22 (42 bp) of the 3′-synthetic-splice-sensor ATP2A1 sequence contains an additional MCS sequence of 12 bp. B) RT-PCR performed on 3′-synthetic-splice-sensor exon 22 mRNA extracted from 3′-synthetic-splice-sensor-V5-MBNLΔ transfected Hek293T cells expressing either low (−dox) or high (+dox) level of MBNL1. 3′-synthetic-splice-sensor that contains a synthetic exon 22, is included only when the level of MBNL1 is high (+dox). C) Western blot analysis showed V5-MBNLΔ protein expression in 3′-synthetic-splice-sensor-V5-MBNLΔ transfected Hek293T cells expressing low level of MBNL1 protein (−dox). V5-MBNLΔ protein expression was reduced by 50% when the level of MBNL1 (+dox) is high in 3′-synthetic-splice-sensor-V5-MBNLΔ transfected Hek293T cells.

FIG. 8. 3′-murine-Splice-sensor-V5-MBNLΔ regulation. A) Schematic representation of the 3′-splice-sensor ATP2A1 sequence inserted between STOP codon and polyA of the V5-MBNLΔ sequence. The splice-sensor is derived from the murine Atp2a1 sequence. B) RT-PCR performed on 3′-splice-sensor Atp2a1 exon 22 mRNA extracted from 3 splice-sensor-V5-MBNLΔ transfected Hek293T cells expressing either low (−dox) or high (+dox) level of MBNL1. 3′-splice-sensor exon 22 is included only when the level of MBNL1 is high (+dox). C) Western blot analysis showed V5-MBNLΔ protein expression in 3′-splice-sensor-V5-MBNLΔ transfected Hek293T cells expressing low level of MBNL1 protein (−dox). V5-MBNLΔ protein expression was reduced by 40% when the level of MBNL1 (+dox) is high in 3′-splice-sensor-V5-MBNLΔ transfected Hek293 T cells.

### DETAILED DESCRIPTION OF THE INVENTION

The present invention relates to a system able to regulate the expression of a product of interest into a cell or a host organism, depending on the activity of a protein regulating alternative splicing within the host cell or tissue. By “activity of a protein regulating alternative splicing” is meant the cellular activity or functionality of the protein existing within the host cell or tissue. By “cellular activity” or “cellular functionality” is meant the ability of the protein regulating alternative splicing to exert its function in the cell. A normal activity corresponds to the activity of the protein regulating alternative splicing, at the physiological, non-pathological state. The activity of the protein regulating alternative splicing within the cell or tissue may be reduced for example as a result of the destruction of said protein, of the sequestration of said protein, of the mutation of the sequence encoding said protein, of the interaction with another molecule inhibiting the function(s) of said protein, or any other event that may negatively affect one or more of the cellular function(s) of said protein. For example, the normal activity of MBNL protein corresponds to the cellular activity of MBNL in a cell of a healthy subject. A reduced or altered or defective activity of MBNL protein may correspond to the cellular activity of MBNL within a cell of a subject having myotonic dystrophy.

The cellular activity of the protein regulating alternative splicing may be determined by analyzing the splicing profile of one or more target(s) of said protein. For example, mis-splicing events in one or more target(s) of the protein may indicate that the cellular activity of the protein regulating alternative splicing is reduced or altered. The skilled person is able to identify mis-splicing events by way of comparison with a normally spliced target, for example from a cell of a healthy patient.

For example, the cellular activity of a MBNL protein may be determined by analyzing the splicing profile of one or more target(s) selected from the non limitative group consisting of: ATP2A1, RYR1, CAPZB, INSR, MBNL1, MBNL2, CLCN1, CAM2B, ITGB, BIN1, TAU or DMD gene.

Alternatively, the skilled person is able to determine, in vitro, the activity of a protein regulating alternative splicing, by transfecting a cell with a DNA or pre-mRNA sequence, that is known to be regulated by the protein regulating alternative splicing, and then analyzing the splicing profile of the corresponding mature transcript.

The present invention thus improves gene therapy in the context of diseases linked to the alteration of the function of a protein regulating alternative splicing. An example of such disease is myotonic dystrophy (DM), wherein the loss-of-function of the MBNL protein results in mis-splicing events leading to symptoms of DM. In the context of DM disease, gene therapy vectors are used to express therapeutic transgene aiming to restore the functional activity of MBNL protein.

The present invention provides a system able to inhibit the expression of a transgene, when a certain level of activity of a protein regulating alternative splicing, such as MBNL, is reached. This is of particular interest, in particular in order to prevent the expression of the transgene within healthy or non-affected tissues or cells, in which the function of the protein regulating alternative splicing is not altered.

Nucleic Acid Molecule

A first aspect of the invention relates to a chimeric nucleic acid molecule comprising:

(i) a first nucleic acid sequence the primary transcript of which comprises an exon, said primary transcript being subject to splicing by a protein regulating alternative splicing; and
(ii) a second nucleic acid sequence comprising a transgene of interest encoding a product of interest;
wherein the protein regulating alternative splicing enhances the inclusion of said exon in the mature transcript of the chimeric nucleic acid molecule, and
wherein said exon is designed to inhibit expression of a functional product of interest, when included into the mature transcript.

The first nucleic acid sequence of the chimeric nucleic acid molecule is herein called “splice-sensor”. As mentioned above, the “splice-sensor” sequence is a nucleic acid sequence whose the primary transcript comprises an exon. The inclusion of said exon in the mature transcript, which is triggered by the protein regulating alternative splicing, leads to the inhibition of the expression of the transgene of interest.

The splice-sensor of the invention is thus able to regulate the expression of the transgene of interest, depending on the endogenous level of activity of said protein regulating alternative splicing:
when the cellular activity of the protein regulating alternative splicing is normal, i.e. when the activity of the protein is not altered: the exon of the splice-sensor is included in the mature transcript of the chimeric molecule thereby inhibiting the expression of the transgene of interest.
when the cellular activity of the protein regulating alternative splicing is defective or reduced: the exon of the splice-sensor is not included in the mature transcript of the chimeric molecule thereby allowing the expression of the transgene of interest.

By “chimeric” nucleic acid molecule is meant a nucleic acid molecule that is not identical to any naturally occurring molecule. The first nucleic acid sequence and the second nucleic acid sequence may derive from two different genes or may derive from the same gene.

Protein Regulating Alternative Splicing

By “protein regulating alternative splicing” is meant any RNA-binding protein that regulates alternative splicing, i.e. any protein that binds to sequence-specific elements in a pre-mRNA to enhance inclusion of alternative exons.

In a particular embodiment, the protein regulating alternative splicing is any protein whose functional defect is associated to a disease.

In a particular embodiment, the protein regulating alternative splicing is selected from the group consisting of: the Muscleblind-like (MBNL) protein, the transactive response DNA-binding protein 43 (TDP-43), the protein fused-in-sarcoma (FUS), the NOVA protein and the RNA binding motif 20 (RBM20).

In a particular embodiment, the protein regulating alternative splicing belongs to the muscleblind-like (MBNL) RNA-binding protein family. In a particular embodiment, the protein regulating alternative splicing is MBNL1, MBNL2 or MBNL3. In a preferred embodiment, the protein regulating alternative splicing is MBNL1.

Splice-Sensor

The term “splice-sensor” as used herein refers to the first nucleic acid sequence of the chimeric nucleic acid molecule, as defined above. The sequence of the splice-sensor may be any natural or synthetic sequence comprising or consisting of a “intron-exon-intron” sequence, wherein the exon able to inhibit the expression and/or activity of the transgene of interest is flanked by two intronic regions.

By “intron” is meant a non-coding sequence within a gene, or its primary transcript, that is removed from the primary transcript and is not present in a corresponding mature messenger RNA molecule.

By “exon” is meant a coding-sequence of a gene that encodes a part of the final mature RNA produced by that gene after introns have been removed by RNA splicing. The term exon refers to both the DNA sequence within a gene and to the corresponding sequence in RNA transcript. In RNA splicing, introns are removed and exons are covalently joined to one another as part of generating the mature messenger RNA.

The exon of the splice-sensor may be any exon able to inhibit the expression of a functional product of interest, when it is included in the mature transcript of the chimeric molecule.

By “inhibiting expression of a functional product of interest”, and declinations thereof, is meant the prevention of the expression of the functional product of interest, or is meant the expression of a non-functional product of interest.

In the context of the present invention, “prevention of the expression of a product of interest” denotes a total or partial inhibition of the expression of the product.

Expression of a non-functional product may be done by a number of alternative means. For example, a non-functional protein of interest can correspond to a truncated form of the protein of interest devoid of one or more portions responsible for one of its property required for its function. Another example of a non-functional protein of interest includes a protein with a destabilized tertiary structure because of the inclusion of a destabilizing portion encoded by the exon of the splice-sensor.

In a particular embodiment, the exon of the splice-sensor is able to inhibit the protein translation of the mRNA transgene, when it is included in the mature transcript of the chimeric molecule. In a particular embodiment, the exon of the splice-sensor comprises one or more “stop codon”. By “stop codon” or “termination codon” is meant a nucleotide triplet within messenger RNA that signals a termination of translation into proteins. In this embodiment, the inclusion of the exon in the mature transcript of the chimeric nucleic acid molecule leads to the occurrence of a premature STOP codon in said mature transcript. Preferably, the exon of the splice-sensor comprises one or more stop codons, such as 1, 2, 3, 4, or more than 4 stop codons.

In another particular embodiment, the exon of the splice-sensor is able to cause a frameshift in the mature transcript of the chimeric nucleic acid molecule, thereby leading to the production of a non-functional product of interest. In a particular embodiment, the inclusion of the exon of the splice-sensor in the mature transcript causes a premature stop codon within said mature transcript.

In another particular embodiment, the exon of the splice-sensor comprises a poly-A tail. In this embodiment, the inclusion of the exon in the mature transcript of the chimeric nucleic acid molecule leads to the occurrence of a premature poly-A tail in said mature transcript, thereby generating a truncated mature transcript.

The inclusion of the exon of the splice-sensor in the mature transcript may alter the translation process by any mechanism. For example, when the exon of the splice-sensor is included after the STOP codon of the transgene of interest, it may induce a nonsense-mediated decay (NMD) or NMD-like mechanism, through an exon junction complex (EJC)-dependent pathway.

In another particular embodiment, the exon of the splice-sensor may comprise a complementary sequence of a microRNA. According to this embodiment, the transgene expression may be inhibited by base-pairing of the microRNA to said complementary sequence, thereby inducing degradation of the mature transcript or preventing the mature transcript from being translated.

In the context of the present invention, the “intron-exon-intron” sequence of the splice-sensor is flanked by two exonic sequences, required for the splicing of the introns contained in the splice-sensor sequence, by the protein regulating alternative splicing. Said exonic sequences may correspond to full exons or may correspond to partial sequences of exons, as long as it provides splice-donor and splice-acceptor sites required for the mRNA splicing of the splice-sensor sequence by the protein regulating alternative splicing.

In a first particular embodiment, the splice-sensor comprising the «intron-exon-intron» sequence is introduced upstream of the sequence encoding the transgene of interest. By “upstream” is meant in 5′ of the sequence encoding the transgene. In a particular embodiment, the splice sensor is introduced upstream of the sequence encoding the transgene of interest but after the start codon. In this embodiment, an exonic region is added at the 5′ end of the splice-sensor and optionally at the 3′ end of the splice-sensor. Preferably, an exonic region is added at the 5′ end of the splice-sensor and at the 3′ end of the splice-sensor.

In a particular embodiment, the splice-sensor is introduced upstream of the sequence encoding the transgene of interest, wherein the transgene of interest encodes a MBNL protein, or a variant thereof, in particular MBNL1, MBNL2 or MBNL3, in particular MBNL1 or a variant thereof.

In a second particular embodiment, the splice-sensor comprising the «intron-exon-intron» sequence is introduced within the sequence encoding the transgene of interest. In a particular embodiment, the splice-sensor is introduced between two exons of the transgene of interest.

In another particular embodiment, the splice-sensor is introduced in the 3′UTR region of the transgene of interest. By 3′UTR region is meant the section of mRNA that immediately follows the translation termination codon. In a particular embodiment, the splice sensor is introduced after the STOP codon of the transgene of interest, in particular between the STOP codon and the polyA sequence of the transgene of interest. The splice-sensor may be introduced anywhere within the sequence encoding the transgene, as long as it is able to inhibit the expression of the transgene.

In a particular embodiment, the splice-sensor is introduced within the sequence encoding the transgene of interest, wherein the transgene of interest encodes in particular a MBNL protein, or a variant thereof, in particular MBNL1, MBNL2 or MBNL3, in particular MBNL1. In a particular embodiment, the splice-sensor is introduced between exon 1 and exon 2, between exon 2 and exon 3, or between exon 3 and exon 4 of MBNL1, MBNL2 or MBNL3 cDNA or a variant thereof, in particular between MBNL1 cDNA or a variant thereof.

In a particular embodiment, the splice-sensor is able to regulate the protein expression of a transgene of interest, depending on the endogenous level of a MBNL protein, such as MBNL1, MBNL2 or MBNL3, in particular MBNL1. In this embodiment, the MBNL protein is responsible for the inclusion of the exon of the splice-sensor into the mature transcript. Consequently, in the presence of MBNL protein, the exon of the splice-sensor is included in the mRNA transcript, thus inhibiting the protein expression of the transgene of interest. On the contrary, in the absence or functional loss of the MBNL protein, the exon of the splice-sensor is not included in the mature mRNA transcript, thus allowing the expression of the transgene of interest. In this embodiment, the splice-sensor comprises or consists of a “intron-exon-intron” sequence, wherein the inclusion of the exon in the mature transcript is triggered by the MBNL protein, in particular by MBNL1.

In a particular embodiment, the splice-sensor is any natural or synthetic sequence that can be bound by a protein regulating alternative splicing, in particular by a MBNL protein, such as MBNL1, MBNL2 or MBNL3, in particular MBNL1. In a particular embodiment, the splice-sensor comprises at least one binding site, which is specifically recognized by the binding domain of the protein regulating alternative splicing. In particular, the splice-sensor may comprise a binding site specifically recognized by a MBNL protein, such as MBNL1, MBNL2 or MBNL3, in particular MBNL1. In a particular embodiment, the binding site comprises any consensus sequence known to be specifically recognized by the protein regulating alternative splicing, in particular by a MBNL protein, such as MBNL1, MBNL2 or MBNL3, in particular MBNL1. In a particular embodiment, the binding site of the splice-sensor comprises a consensus sequence consisting of a GpC dinucleotide flanked by pyrimidines, i.e. “YGCY” (Y being uridine or cytosine).

In a particular embodiment, the splice-sensor is a sequence that is bound by a MBNL protein such as MBNL1, MBNL2 or MBNL3, in particular MBNL1. Said sequence bound by a MBNL protein can derive from any target gene of MBNL, in particular MBNL1, MBNL2 or MBNL3, in particular MBNL1.

In a particular embodiment, the splice-sensor is a sequence that derives from a gene containing an exon regulated by a MBNL protein. In a particular embodiment, the splice-sensor is a sequence that derives from ATP2A1, RYR1, CAPZB, INSR, CAM2B, ITGB, BIN1, TAU or DMD gene.

In a particular embodiment, the splice-sensor is a synthetic sequence. By “synthetic sequence” is meant either a natural sequence that is modified by deletion(s), substitution(s), addition(s) of one or more nucleotide(s) or a fully synthetic sequence not based on a natural sequence. The synthetic splice-sensor may be any intron-exon-intron sequence comprising: (i) the elements required for the binding and splicing activity of the protein regulating alternative splicing, such as MBNL; and (ii) an exon able to inhibit the expression of the functional product of interest, when it is included in the mature transcript of the chimeric molecule.

In a particular embodiment, the splice-sensor is designed so that it can fit, in combination with a transgene of interest, in a vector, such as a plasmid or viral vector. In particular, the splice-sensor is designed so that it can fit, in combination with a transgene of interest, into an AAV vector. A person skilled in the art is able to adapt the sequence and size of the splice-sensor, depending on the size of the transgene of interest and on the packaging capacity of the vector used for expressing the transgene of interest.

In a particular embodiment, the splice-sensor is a sequence that derives from the ATP2A1 gene.

In a particular embodiment, the exon of the splice-sensor is exon 22 of the ATP2A1 gene, exon 70 of the RYR1 gene, exon 8 of the CAPZB gene, exon 11 of the INSR gene, exon 13 of the CAM2B gene, exon 17 of the ITGB gene, exon 11 of the BIN1 gene, exon 2 or 3 of the TAU gene or exon 78 of the DMD gene.

In a particular embodiment, the exon of the splice-sensor is exon 22 of the ATP2A1 gene encoding the Sarcoplasmic/endoplasmic reticulum calcium ATPase 1. Indeed, the inclusion of exon 22 of ATP2A1 gene in the corresponding pre-mRNA transcript is regulated by MBNL1. In addition, exon 22 of ATP2A1 gene contains two stop codons. Said stop codons are thus able to inhibit the protein translation of the transgene, when exon 22 is included in the mature transcript of the chimeric molecule.

In a particular embodiment, the splice-sensor comprises exon 22 of the ATP2A1 gene, flanked by intron 21 and intron 22 of the ATP2A1 gene. In particular, the splice-sensor may comprise or consist of the sequence “intron 21-exon 22-intron 22” of the ATP2A1 gene. More particularly, the sequence “intron 21-exon 22-intron 22” of the ATP2A1 gene may be further flanked by exonic regions, in particular by exonic regions derived from exon 21 and exon 23 of the ATP2A1 gene.

In a particular embodiment, the splice-sensor comprises or consists of the following sequence: 3′ end of exon 21-intron 21-exon 22-intron 22-5′ end of exon 23. In a particular embodiment, the “3′ end of exon 21” comprises a partial sequence of the exon 21 joining the 5′ end of intron 21. In a particular embodiment, the “3′ end of exon 21” correspond to the sequence having at least 10, 20, 30, 40, 50 nucleotides from the 3′ end of exon 21, in particular at least 30 nucleotides, in particular around 34 nucleotides from the 3′ end of exon 21.

The “5′end of exon 23” comprises a partial sequence of the exon 23 joining the 3′ end of intron 22. In a particular embodiment, the “5′end of exon 23” comprises at least 10, 20, 30, 40, 50 nucleotides from the 5′ end of exon 23, in particular at least 20 nucleotides, in particular around 22 nucleotides from the 5′ end of exon 23.

In a particular embodiment, the splice-sensor comprises or consist of the sequence as shown in SEQ ID NO: 9. In a particular embodiment, the splice-sensor comprises or consists of a sequence having at least 50%, in particular at least 60%, 70%, 80%, 90% and more particularly at least 95% or even at least 99% identity to the sequence of SEQ ID NO: 9.

In a particular embodiment, the splice-sensor comprises or consist of the sequence as shown in SEQ ID NO: 29. In a particular embodiment, the splice-sensor comprises or consists of a sequence having at least 50%, in particular at least 60%, 70%, 80%, 90% and more particularly at least 95% or even at least 99% identity to the sequence of SEQ ID NO: 29.

In a particular embodiment, the splice-sensor comprises or consist of the sequence as shown in SEQ ID NO: 30. In a particular embodiment, the splice-sensor comprises or consists of a sequence having at least 50%, in particular at least 60%, 70%, 80%, 90% and more particularly at least 95% or even at least 99% identity to the sequence of SEQ ID NO: 30.

In a particular embodiment, the splice-sensor comprises or consist of the sequence as shown in SEQ ID NO: 31. In a particular embodiment, the splice-sensor comprises or consists of a sequence having at least 50%, in particular at least 60%, 70%, 80%, 90% and more particularly at least 95% or even at least 99% identity to the sequence of SEQ ID NO: 31.

Transgene of Interest

As described above, the chimeric nucleic acid molecule of the invention comprises a nucleic acid sequence comprising a transgene of interest, the expression of said transgene of interest being regulated by the splice-sensor sequence as described above.

In a particular embodiment, the transgene of interest is a cDNA sequence.

The transgene of interest may encode any therapeutic product of interest able to treat a disease linked to a defect in the function of a protein regulating alternative splicing.

In a particular embodiment, the product of interest is able to restore or compensate the loss of function of the protein regulating alternative splicing.

In a particular embodiment, the transgene of interest encodes the protein regulating alternative splicing as defined above, or a variant thereof. In this embodiment, the expression of the protein regulating alternative splicing is auto-regulated, depending on the endogenous level of the protein regulating alternative splicing. In other words, the expression of the protein regulating alternative splicing is inhibited, when normal and/or functional level of the protein regulating alternative splicing is reached.

In a particular embodiment, the product of interest is able to restore the function of the MBNL protein, such as MBNL1, MBNL2 or MBNL3, in particular of MNBL1 protein.

In a particular embodiment, the product of interest is able to act directly or not on the behavior of CUGexp-RNA or MBNL activity.

In a particular embodiment, the product of interest is able to restore the function of the MBNL protein by degrading pathological CTG expansions or CUG-expansions responsible for the sequestration of endogenous MBNL. In yet another particular embodiment, the product of interest is able to restore the function of the MBNL protein by removing/reducing pathological CTG expansions, or inhibiting transcription of pathological CTG expansions or the DMPK gene containing these expansions, or degrading or interfering with CUG-expansions responsible for the sequestration of endogenous MBNL. The product of interest may also be a protein product such as natural or synthetic RNA binding proteins, CRISPR/CAS derivates or an RNA product such as shRNA, miRNA, sgRNA, U7-snRNA targeting said pathological CUG-expansions. In a particular embodiment, the product of interest is an endonuclease such as a Cas9 endonuclease. In a particular embodiment, an RNA-targeting Cas9 (RCas9) able to eliminate CUG repeats may be used. The product of interest may be a dCas9-eGFP, for example as described by Nelles et al. (Nelles et al., 2016). The product of interest may be a dCas9 fused to an PIN-endonuclease, as developed by Batra et al. (Batra et al., 2017).

In a particular embodiment, the transgene of interest encodes a dCas9-eGFP peptide or variant thereof having a sequence at least 50%, in particular at least 60%, 70%, 80%, 90% and more particularly at least 95% or even at least 99% identical to the amino acid sequence of SEQ ID NO:27. In a particular embodiment, the transgene of interest encodes a dCas9-eGFP protein comprising or consisting of the amino acid sequence of SEQ ID NO: 27.

In a particular embodiment, the nucleic acid molecule of the invention comprises:
a first nucleic acid sequence, i.e. the “splice-sensor” as described herein, which is bound by a MBNL protein, such as MBNL1, MBNL2 or MBNL3, in particular MBNL1; and
a second nucleic acid sequence encoding a Cas9 endonuclease, such as the nucleic acid sequence of SEQ ID NO: 28, encoding the dCas9-eGFP peptide of SEQ ID NO: 27.

In a particular embodiment, the nucleic acid molecule of the invention comprises or consists of the sequence as shown in SEQ ID NO:21.

In a particular embodiment, when the exon of the splice-sensor is not included in the mature transcript of the chimeric molecule, the protein encoded by said mature transcript consists of the sequence as shown in SEQ ID NO: 22.

In the context of myotonic dystrophy disease, previous gene therapies aimed at providing to the treated cell overexpression of a MBNL protein that will compensate for the loss of free and functionally available endogenous MBNL proteins which have been sequestered onto pathological CUG-repeats. Thus, in a particular embodiment, the transgene of interest encodes a MBNL protein, or a variant thereof, in particular MBNL1, MBNL2 or MBNL3, in particular MBNL1.

In a particular embodiment, the transgene of interest encodes a MBNL1 peptide or variant thereof having a sequence at least 50%, in particular at least 60%, 70%, 80%, 90% and more particularly at least 95% or even at least 99% identical to the amino acid sequence of SEQ ID NO: 1. In a particular embodiment, the transgene of interest encodes a MBNL1 protein comprising or consisting of the amino acid sequence of SEQ ID NO: 1.

In a particular embodiment, the transgene of interest encodes a MBNL2 peptide or variant thereof having a sequence at least 50%, in particular at least 60%, 70%, 80%, 90% and more particularly at least 95% or even at least 99% identical to the amino acid sequence of SEQ ID NO: 2. In a particular embodiment, the transgene of interest encodes a MBNL2 protein comprising or consisting of the amino acid sequence of SEQ ID NO: 2.

In a particular embodiment, the transgene of interest encodes a MBNL3 peptide or variant thereof having a sequence at least 50%, in particular at least 60%, 70%, 80%, 90% and more particularly at least 95% or even at least 99% identical to the amino acid sequence of SEQ ID NO: 3. In a particular embodiment, the transgene of interest encodes a MBNL3 protein comprising or consisting of the amino acid sequence of SEQ ID NO: 3.

In another particular embodiment, the transgene of interest encodes a “modified MBNL polypeptide”. The patent application WO2015/158365 describes a gene therapy based on the expression of a modified MBNL protein, i.e. a non-functional variant MBNL protein having reduced or even no splicing activity, to compete, displace, and replace thereon endogenous MBNL protein(s) to avoid the negative consequences of their sequestration. As provided in the experimental part of the disclosure of the patent application WO2015/158365, the results obtained with this strategy have been extremely satisfying. The present invention improves said therapeutic strategy, in providing a system able to regulate the expression of the “modified MBNL polypeptide”, depending on the level of endogenous MBNL protein(s).

In a particular embodiment, the modified MBNL polypeptide has a reduced splicing activity, when compared to splicing activity of full-length MBNL protein but maintains its YGCY binding property. In particular, said modified MBNL polypeptide may be able to bind pathological CUG repeat. Moreover, the modified MBNL polypeptide used herein can counteract the CUGexp-RNA toxicity by releasing sequestered endogenous MBNL from the CUGexp-RNA aggregates in order to restore the function of these endogenous MBNL protein.

In a particular embodiment, the modified MBNL polypeptide is able to bind the MBNL YGCY RNA-motif, with “Y” representing a pyrimidine (uridine or cytosine). In particular, the modified MBNL polypeptide may be able to bind UGCU-motif, which is the building block of the pathological DM1 expanded CUG repeats. In a particular embodiment, the modified MBNL polypeptide includes or not the amino acids corresponding to exon 3 of the MBNL1 mRNA (accession number NM_021038). In a further embodiment, the modified MBNL polypeptide lacks the amino acids of SEQ ID NO: 4 (SEQ ID NO: 4: TQ SAVKSLKRPLEATFDLGIPQAVLPPLPKRPALEKTNGATAVFNTGIFQYQQALANM QLQQHTAFLPPGSILCMTPATSVVPMVHGATPATVSAATTSATSVPFAATTANQIPIIS AEHLTSHKYVTQM) corresponding to exons 5 to 10 of the MBNL1 mRNA.

As used herein, the term “MBNL” denotes all paralogue members of the muscleblind-like RNA-binding protein family and includes in particular MBNL1, -2 and -3. In a particular embodiment, the modified MBNL polypeptide is derived from the human MBNL1 protein sequence. In an embodiment, the modified MBNL polypeptide is a MBNL1 protein having the exon 3 encoded sequence but lacking the amino acid sequences encoded by exons 5 to 10 of the MBNL1 gene. In a specific embodiment, the modified MBNL1-derived polypeptide is referred to as “MBNLΔ” having the following amino acid sequence:

(SEQ ID NO: 5)
MAVSVTPIRDTKWLTLEVCREFQRGTCSRPDTECKFAHPSKSCQVENGRV
IACFDSLKGRCSRENCKYLHPPPHLKTQLEINGRNNLIQQKNMAMLAQQM
QLANAMMPGAPLQPVPMFSVAPSLATNASAAAFNPYLGPVSPSLVPAEIL
PTAPMLVTGNPGVPVPAAAAAAAQKLMRTDRLEVCREYQRGNCNRGENDC
RFAHPADSTMIDTNDNTVTVCMDYIKGRCSREKCKYFHPPAHLQAKIKAA
QYQVNQAAAAQAAATAAAM.

In an embodiment, the modified MBNL polypeptide is a MBNL1 protein lacking both the exon 3 encoded sequence and the sequences encoded by exons 5 to 10 of the MBNL1 gene. In a specific embodiment, the non-functional MBNL1-derived polypeptide is referred to as MBNLΔCT having the following amino acid sequence:

(SEQ ID NO: 6)
MAVSVTPIRDTKWLTLEVCREFQRGTCSRPDTECKFAHPSKSCQVENGRV
IACFDSLKGRCSRENCKYLHPPPHLKTQLEINGRNNLIQQKNMAMLAQQM
QLANAMMPGAPLQPVVCREYQRGNCNRGENDCRFAHPADSTMIDTNDNTV
TVCMDYIKGRCSREKCKYFHPPAHLQAKIKAAQYQVNQAAAAQAAATAAA
M.

In another embodiment, the modified MBNL polypeptide is a MBNL2 protein encoded by the amino acid sequences of exons 2 to 5 of the MBNL2 protein. In a specific embodiment, the modified MBNL2-derived polypeptide is referred to as MBNL2-ACT3 having the following amino acid sequence:

(SEQ ID NO: 7)
MALNVAPVRDTKWLTLEVCRQFQRGTCSRSDEECKFAHPPKSCQVENGRV
IACFDSLKGRCSRENCKYLHPPTHLKTQLEINGRNNLIQQKTAAAMLAQQ
MQFMFPGTPLHPVPTFPVGPAIGTNTAISFAPYLAPVTPGVGLVPTEILP
TTPVIVPGSPPVTVPGSTATQKLLRTDKLEVCREFQRGNCARGETDCRFA
HPADSTMIDTSDNTVTVCMDYIKGRCMREKCKYFHPPAHLQAKIKAAQHQ
ANQAAVAAQAAAAAATVM.

In another embodiment, the modified MBNL polypeptide is a MBNL3 protein encoded by the amino acid sequences of exons 1 to 4 of the MBNL3 protein. In a specific embodiment, the modified MBNL3-derived polypeptide is referred to as MBNL3-ACT3 having the following amino acid sequence:

(SEQ ID NO: 8)
MTAVNVALIRDTKWLTLEVCREFQRGTCSRADADCKFAHPPRVCHVENGR
VVACFDSLKGRCTRENCKYLHPPPHLKTQLEINGRNNLIQQKTAAAMFAQ
QMQLMLQNAQMSSLGSFPMTPSIPANPPMAFNPYIPHPGMGLVPAELVPN
TPVLIPGNPPLAMPGAVGPKLMRSDKLEVCREFQRGNCTRGENDCRYAHP
TDASMIEASDNTVTICMDYIKGRCSREKCKYFHPPAHLQARLKAAHHQMN
HSAASAM.

As used herein a “variant” of the modified MBNL polypeptide of the invention is a protein having the same or similar binding properties to the YGCY motif, in particular to CUG repeats, as the wild-type MBNL protein it is derived from (in particular MBNL1, 2 or 3) or as the modified MBNL protein of SEQ ID NO: 5, SEQ ID NO: 6, SEQ ID NO: 7 or SEQ ID NO: 8 as shown above, and wherein said variant has a reduced splicing activity as compared to the wild-type MBNL protein. In other terms, the modified MBNL polypeptide used in the present invention is a non-functional MBNL polypeptide, that has low or even no splicing activity as compared to the wild-type parent MBNL protein.

In a particular embodiment, the variant according to the invention may have a sequence at least 50%, in particular at least 60%, 70%, 80%, 90% and more particularly at least 95% or even at least 99% identical to the amino acid sequence corresponding to exons 1 to 4 of the wild-type MBNL protein (e.g. of MBNL1, 2 or 3) or to the amino acid sequence shown in SEQ ID NO: 5, 6, 7, or 8. In a particular embodiment, the transgene of interest encodes a sequence having at least 50%, in particular at least 60%, 70%, 80%, 90% and more particularly at least 95% or even at least 99% identity to the amino acid sequence of SEQ ID NO: 5.

The modified MBNL polypeptide has almost no splicing activity, or otherwise said as a reduced activity, as compared to wild-type MBNL protein. By “almost no activity” or “reduced activity”, it is herein intended to describe a splicing activity, which is reduced by at least 50%, in particular by at least 60%, 70%, 75%, 80%, 85%, 90% or even at least 95% as compared to the splicing activity of wild-type MBNL protein. Such activity may be determined according to methods well known by those skilled in the art such as the use of minigenes to analyze the alternative splicing of cTNT exon 5, IR exon 11 and Tau exon 2 (Tran et al., 2011).

In a particular embodiment, the modified MBNL protein may comprise a localization sequence such as a nuclear localization sequence (NLS) or a nuclear export signal (NES). A representative NLS has the sequence represented in SEQ ID NO: 10: PKKKRKV. A representative NES has the sequence represented in SEQ ID NO: 11: LPPLERLTLD. The present disclosure includes any modified MBNL polypeptide as described above, combined with such a localization sequence, in particular with an NLS or NES such as those specifically mentioned above.

In a particular embodiment, the transgene of interest encodes a product of interest, such as a MBNL protein or a variant thereof, or a modified MBNL protein as defined above, that is fused to a peptide tag. In a particular embodiment, the tag is a V5 tag, in particular a V5 tag having the sequence as shown in SEQ ID NO: 12.

In a particular embodiment, the transgene of interest comprises or consists of the sequence as shown in SEQ ID NO: 17 or SEQ ID NO: 18, in particular SEQ ID NO: 17. In a particular embodiment, the transgene of interest comprises or consists of a sequence having at least 50%, in particular at least 60%, 70%, 80%, 90% and more particularly at least 95% or even at least 99% identity to the sequence of SEQ ID NO: 17 or SEQ ID NO: 18, in particular SEQ ID NO: 17.

In a particular embodiment, the transgene of interest encodes a protein comprising or consisting of the sequence as shown in SEQ ID NO: 5 or SEQ ID NO: 13, in particular SEQ ID NO: 5.

In a particular embodiment, the transgene of interest encodes a protein comprising or consisting of a sequence having at least 50%, in particular at least 60%, 70%, 80%, 90% and more particularly at least 95% or even at least 99% identity to the sequence of SEQ ID NO: 5 or SEQ ID NO: 13, in particular SEQ ID NO: 5.

In a particular embodiment, the nucleic acid molecule of the invention comprises:
a first nucleic acid sequence, i.e. the “splice-sensor”, which is bound by a MBNL protein, such as MBNL1, MBNL2 or MBNL3, in particular MBNL1; and
a second nucleic acid sequence encoding the modified MBNL peptide as defined above, in particular the MBNLΔ as defined above.

In a particular embodiment, the nucleic acid molecule of the invention comprises:
a first nucleic acid sequence, i.e. the “splice-sensor”, comprising the exon 22 of the ATP2A1 gene, in particular the exon 22 of the ATP2A1 gene flanked by intron 21 and intron 22 of the ATP2A1 gene; and
a second nucleic acid sequence encoding the modified MBNL peptide as defined above, in particular the MBNLΔ as defined above.

In a particular embodiment, the nucleic acid molecule of the invention comprises:
a first nucleic acid sequence, i.e. the “splice-sensor”, comprising or consisting of the sequence “3′ end of exon 21-intron 21-exon 22-intron 22-5′ end of exon 23”, as defined above; and
a second nucleic acid sequence encoding the modified MBNL peptide as defined above, in particular the MBNLΔ as defined above.

In a particular embodiment, the nucleic acid molecule of the invention comprises a first nucleic acid sequence, i.e. the “splice-sensor” upstream of the second nucleic acid sequence encoding the modified MBNL peptide as defined above, in particular the MBNLΔ as defined above.

In a particular embodiment, the nucleic acid molecule of the invention comprises or consists of the sequence as shown in SEQ ID NO: 14 or SEQ ID NO: 19.

In a particular embodiment, the nucleic acid molecule of the invention comprises or consists of a sequence having at least 50%, in particular at least 60%, 70%, 80%, 90% and more particularly at least 95% or even at least 99% identity to the sequence of SEQ ID NO: 14 or SEQ ID NO: 19.

In a particular embodiment, when the exon of the splice-sensor is not included in the mature transcript of the chimeric molecule, the protein encoded by said mature transcript consists of the sequence as shown in SEQ ID NO: 15 or SEQ ID NO: 20.

In another particular embodiment, when the exon of the splice-sensor is included in the mature transcript of the chimeric molecule, the protein encoded by said transcript consists of the sequence as shown in SEQ ID NO: 16.

In another particular embodiment, the nucleic acid molecule of the invention comprises a first nucleic acid sequence (i.e. the “splice-sensor” as described herein) inserted between 2 exons of the second nucleic acid sequence encoding the modified MBNL peptide as defined above, in particular the MBNLΔ as defined above.

In a particular embodiment, the splice-sensor is inserted between exon 2 and exon 3 of the nucleic acid sequence encoding the modified MBNL peptide as defined above, in particular the MBNLΔ as defined above. In a particular embodiment, the nucleic acid molecule of the invention comprises or consists of the sequence as shown in SEQ ID NO: 23. In a particular embodiment, the nucleic acid molecule of the invention comprises or consists of a sequence having at least 50%, in particular at least 60%, 70%, 80%, 90% and more particularly at least 95% or even at least 99% identity to the sequence of SEQ ID NO: 23.

In a particular embodiment, the splice-sensor is inserted between exon 3 and exon 4 of the nucleic acid sequence encoding the modified MBNL peptide as defined above, in particular the MBNLΔ as defined above. In a particular embodiment, the nucleic acid molecule of the invention comprises or consists of the sequence as shown in SEQ ID NO: 24. In a particular embodiment, the nucleic acid molecule of the invention comprises or consists of a sequence having at least 50%, in particular at least 60%, 70%, 80%, 90% and more particularly at least 95% or even at least 99% identity to the sequence of SEQ ID NO: 24.

In another particular embodiment, the nucleic acid molecule of the invention comprises a first nucleic acid sequence (i.e. the “splice-sensor” as described herein) inserted after the STOP codon of the second nucleic acid sequence encoding the modified MBNL peptide as defined above, in particular the MBNLΔ as defined above. In a particular embodiment, the “splice-sensor” is inserted between the STOP codon and the polyA of the second nucleic acid sequence encoding the modified MBNL peptide as defined above, in particular the MBNLΔ as defined above. In a particular embodiment, the nucleic acid molecule of the invention comprises or consists of the sequence as shown in SEQ ID NO: 25 or SEQ ID NO: 26. In a particular embodiment, the nucleic acid molecule of the invention comprises or consists of a sequence having at least 50%, in particular at least 60%, 70%, 80%, 90% and more particularly at least 95% or even at least 99% identity to the sequence of SEQ ID NO: 25 or SEQ ID NO:26.

Expression Cassette

Another aspect of the invention relates to an expression cassette comprising or consisting of the nucleic acid molecule as described above, operably linked to regulatory sequences, (such as suitable promoter(s), enhancer(s), terminator(s), etc. . . . ) allowing the expression (e.g. transcription and translation) of the nucleic acid molecule as described above, in a host cell. The expression cassette of the invention may be DNA or RNA, and is preferably double-stranded DNA. The expression cassette of the invention may also be in a form suitable for transformation of the intended host cell or host organism, in a form suitable for integration into the genomic DNA of the intended host cell or in a form suitable for independent replication, maintenance and/or inheritance in the intended host organism.

For instance, the expression cassette of the invention may be in the form of a vector, such as for example a plasmid, cosmid, YAC, a viral encoding vector or transposon. In particular, the vector may be an expression vector, i.e. a vector that can provide for expression in vitro and/or in vivo (e.g. in a suitable host cell, host organism and/or expression system). In a preferred but non-limiting aspect, an expression cassette of the invention comprises i) the chimeric nucleic acid molecule of the invention; operably linked to ii) one or more regulatory elements, such as a promoter and optionally a suitable terminator; and optionally also iii) one or more further elements of genetic constructs such as 3′- or 5′-UTR sequences, leader sequences, selection markers, expression markers/reporter genes, and/or elements that may facilitate or increase (the efficiency of) transformation or integration or subcellular localization or expression of the transgene of interest, such as nuclear localization signal (NLS) or nuclear export signal (NES).

Vector

Another aspect of the invention relates to a vector comprising the nucleic acid molecule of described above or the expression cassette as described above.

In particular, said vector may be a plasmid or a viral vector. Suitable viral vectors used in practicing the present invention include retroviruses, lentiviruses, adenoviruses and adeno-associated viruses. In a particular embodiment, the invention relates to a lentivirus comprising a nucleic acid sequence molecule according to the invention. In another particular embodiment, the invention relates to an AAV vector, in particular an AAV1, AAV2, AAV3, AAV4, AAV5, AAV6, AAV7, AAV8, AAV9, AAV10 or AAV11 vector, in particular an AAV9 vector, comprising a nucleic acid sequence molecule according to the invention. The AVV vector may be a pseudotyped vector, i.e. its genome and its capsid may be derived from different AAV serotypes. For example, the genome may be derived from an, AAV2 genome and its capsid proteins may be of the AAV1, AAV3, AAV4, AAV5, AAV6, AAV7, AAV8, AAV9, AAV10 or AAV11 serotype.

Cell

Another aspect of the invention relates to an isolated cell transformed with the nucleic acid molecule, the expression cassette or the vector as described above.

Methods and Uses

Another aspect of the invention relates to the nucleic acid molecule, the expression cassette, the vector, or the isolated cell as described above, for use as a medicament.

In a particular embodiment, the nucleic acid molecule, the expression cassette, the vector or the isolated cell of the invention are used in the treatment of a disease or disorder that can benefit from the expression of the transgene of interest. In a particular embodiment, the nucleic acid molecule, the expression cassette, the vector, or the isolated cell as described above are useful therapeutic agents, in the treatment of a disease linked to a defect in the function of a protein regulating alternative splicing.

In a particular embodiment, the nucleic acid molecule, the expression cassette, the vector, or the isolated cell as described above are used in the treatment of a disease or disorder linked to a defect in the function of the transactive response DNA-binding protein 43 (TDP-43) or the protein fused-in-sarcoma (FUS), such as in the treatment of Amyotrophic Lateral Sclerosis.

In a particular embodiment, the nucleic acid molecule, the expression cassette, the vector, or the isolated cell as described above is uses in the treatment of a disease or disorder linked to a defect in the function of the NOVA protein, such as in the treatment of a paraneoplastic neurological disorder.

In a particular embodiment, the nucleic acid molecule, the expression cassette, the vector, or the isolated cell as described above is used in the treatment of a disease or disorder linked to a defect in the function of the RNA binding motif 20 (RBM20), such as in the treatment of Dilated Cardiomyopathy.

In a particular embodiment, the nucleic acid molecule, the expression cassette, the vector, or the isolated cell as described above are useful therapeutic agents in the treatment of a disease or disorder linked to a sequestration of a MBNL protein, or to a deregulated function of a MBNL member such as MBNL1, or other paralogue members (including MBNL2 and MBNL3). In a preferred embodiment, the modified polypeptide of the invention is used for the treatment of a myotonic dystrophy such as DM1 and DM2, or any disease where a loss of MBNL function (e.g. sequestration, aggregation, mutations . . . ) may be rescued by ectopic delivery of a functional MBNL polypeptide or a modified MBNL polypeptide as defined above.

In a further aspect, the invention relates the nucleic acid molecule, the expression cassette, the vector, or the isolated cell as described above, for use in a method for the treatment of a myotonic dystrophy.

As used herein, the term “treatment” or “therapy” includes curative and/or preventive treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Preventive treatment refers to any of: halting the onset, delaying the onset, reducing the development, reducing the risk of development, reducing the incidence, reducing the severity, as well as increasing the time to onset of symptoms and survival for a given disorder.

It is thus described a method for treating a disease linked to a defect in the function of a protein regulating alternative splicing, such as myotonic dystrophies, in a subject in need thereof, which method comprises administering said patient with the nucleic acid molecule, the expression cassette, the vector, or the isolated cell of the invention.

Within the context of the invention, “subject” or “patient” means a mammal, particularly a human, whatever its age or sex, suffering of a myotonic dystrophy. The term specifically includes domestic and common laboratory mammals, such as non-human primates, felines, canines, equines, porcines, bovines, goats, sheep, rabbits, rats and mice. Preferably the patient to treat is a human being, including a child or an adolescent.

For the uses and methods according to the invention, the nucleic acid molecule, the expression cassette, the vector, or the isolated cell may be formulated by methods known in the art. In addition, any route of administration may be envisioned. For example, the nucleic acid molecule, the expression cassette, the vector, or the isolated cell may be administered by any conventional route of administration including, but not limited to oral, pulmonary, intraperitoneal (ip), intravenous (iv), intramuscular (im), subcutaneous (sc), transdermal, buccal, nasal, sublingual, ocular, rectal and vaginal. In addition, administration directly to the nervous system may include, and are not limited to, intracerebral, intraventricular, intracerebroventricular, intrathecal, intracistemal, intraspinal or peri-spinal routes of administration by delivery via intracranial or intravertebral needles or catheters with or without pump devices. It will be readily apparent to those skilled in the art that any dose or frequency of administration that provides the therapeutic effect described herein is suitable for use in the present invention. In a particular embodiment, the subject is administered a viral vector encoding a modified MBNL polypeptide according to the invention by the intramuscular route. In a specific variant of this embodiment, the vector is an AAV vector as defined above, in particular an AAV9 vector. In a further specific aspect, the subject receives a single injection of the vector.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate.

Another aspect of the invention relates to the nucleic acid molecule, the expression cassette, the vector, or the isolated cell as described above, for use in a method for controlling the expression of the transgene of interest, depending on the endogenous level of the protein regulating alternative splicing.

The below examples illustrate the invention without limiting its scope.

### EXAMPLES

Material and Methods

Constructs

The splice-sensor described in examples 1 and 2 was derived from the human ATP2A1 gene and contains partial exon 21 (34 nucleotides from the 3′-end of the intron), intron 21, exon22, intron 22 and partial exon 23 (22 nucleotides from the 5′-end of the intron). This splice-sensor ATP2A1 exon22 was inserted in front of either an EGFP or a V5-MBNLΔ sequence into a pcDNA3.1(+) expression vector. Both constructs were verified by sequencing. MBNLΔ containing amino acids from 1 to 269 has been derived from MBNL1, tagged to V5 protein and cloned into pcDNA3.1(+) or pSMD2 vectors.

In example 3, the same splice sensor derived from the human ATP2A1 gene was inserted upstream of a sequence encoding dCas9-eGFP. The dCas9-eGFP plasmid was obtained from Addgen (#74710)

In example 4, a splice sensor comprising int21-ex22-int22 of the human ATP2A1 gene is inserted between exons 2 and 3, or between exons 3 and 4 of the V5-MBNLΔ sequence. An extra base (T) was inserted inside the ex22 to keep the 2 Stop codons within ex22 in frame.

In example 5, a multiple cloning site sequence (12 bp) was inserted into exon22 (42 bp). The splice sensor comprises partial exon 21 (34 nucleotides from the 3′-end of the intron), intron 21, exon 22+MCS(54 bp), intron 22 and partial exon 23 (23 nucleotides from the 5′-end of the intron). In example 5, this splice sensor is inserted just after the STOP codon of V5-MBNLΔ (between STOP codon and polyA sequence).

In example 6, the splice sensor is derived from the murine ATP2A1 gene and contains partial exon 21 (50 nucleotides from the 3′-end of the intron), intron 21, exon22, intron 22 and full exon 23 (268 nucleotides from the 5′-end of the intron). In example 6, this splice sensor is inserted just after the STOP codon of V5-MBNLΔ (between STOP codon and polyA sequence).

Cell Culture and Transfection

A HEK293 T-Rex FLP stable cell line containing tetracycline-inducible full length MBNL1 protein with an N-terminal tag was used. Cells were routinely cultured as a monolayer in Dulbecco's modified Eagle's medium (DMEM)-GlutaMax (Invitrogen) supplemented with 10% fetal bovine serum (Gibco) at 37° under 5% CO2. Prior to transfection, cells were seeded in twenty-four-well or twelve-well plates at density 1.6×105 cells/well. Cells were transfected 24 h later at approximately 80% of confluence. Plasmid (20 ng/well) was transfected into each well using Lipofectamine LTX Reagent (Invitrogen) following the manufacturer's protocol. After 4 h the medium was changed to DMEM supplemented with 5% fetal bovine serum and containing doxycycline at the concentration 1 μg/ml (Sigma-Aldrich). Cells were harvested 48 h post-transfection.

In Vivo Gene Transfer

All mouse procedures were done according to the protocol n° 01204.02 approved by the Ethical Committee on Animal Resources at the Centre Experimentation Fonctionnelle of Pitie-Salpetriere animal facility and under appropriate biological containment. Adult control WT (FVB) or DM1 (HSALR) transgenic mice were intramuscularly injected with saline or AAV9 vectors and sacrificed seven weeks after. AAV9-V5-MBNLΔ or AAV9-splice-sensor-V5-MBNLΔ were produced by the AAV core facility of the Centre de Recherche en Myologie.

RNA Isolation and RT-PCR

Total RNA was isolated from cells or muscles using TRI Reagent (Sigma) and 1 ug of total RNA was reverse transcribed using M-MLV first-strand synthesis system (Life technologies) in a total of 20 μl. Samples were treated with RQ1 DNase (Promega) and lul of cDNA preparation was used in a semi-quantitative PCR analysis according to standard protocol (ReddyMix, Thermo Scientific). PCR amplification was carried out for 30 cycles at 60° and specific primers.

Western Blotting

HEK cell pellets or muscle tissues were lysed in RIPA buffer (150 mM NaCl, 50 mM Tris-HCl pH 8, 0.1 mM ethylenediamine tetra-acetic acid (EDTA), 1% NP-40, 0.5% sodium dodecyl sulfate (SDS)) supplemented with Complete Protease Inhibitor Cocktail (Roche)). Lysates were sonicated at 4.0 and centrifuged at 14 000×g for 10 min at 4° C. Protein concentration was determined with the BCA protein assay kit (Thermo scientific). Samples were diluted in Laemmli Reducing Sample Buffer supplemented with 50 mM DTT, heated to 70.0 for 5 min, separated on 4-12% Bis-Tris polyacrylamide gels (Thermo scientific) and transferred to nitrocellulose membrane (Porablot NCP, Macherey-Nagel) using a wet transfer apparatus (1 h, 100 V, 4° C.). Membrane were blocked for 1 h in 5% skim milk in PBST buffer (phosphate buffered saline (PBS), 0.1% Tween-20) and incubated with a primary antibody against MBNL1 (MBla 1:1000), V5 (1:1000, Invitrogen) GFP (1:1000, Clontech) or GAPDH (1:1000, Santa Cruz). Anti-rabbit (1:20 000, Invitrogen) and anti-mouse (1:2000, Invitrogen) secondary antibodies were conjugated with horseradish peroxidase and detected using the Immobilon Western Chemiluminescent HRP Substrate system (EMD Millipore). Alternatively, total-protein normalization was used, using the Stain-Free technology (Biorad).

Results

The present invention relates to a nucleic acid molecule comprising a “splice-sensor” that regulates the expression of a transgene of interest according to the activity of a specific protein regulating alternative splicing within the host cell or tissue.

As an example, we took advantage of the DM1 paradigm in which mutant transcripts containing expanded CUG repeats sequester MBNL protein leading to a functional loss of MBNL splicing factor and subsequently, to splicing misregulation associated with symptoms. In this context, a splice-sensor that is regulated by MBNL1 splicing factor was developed in order to regulate the expression of a transgene, depending on the level of MBNL1 activity.

### Example 1: Splice-Sensor-GFP Regulation

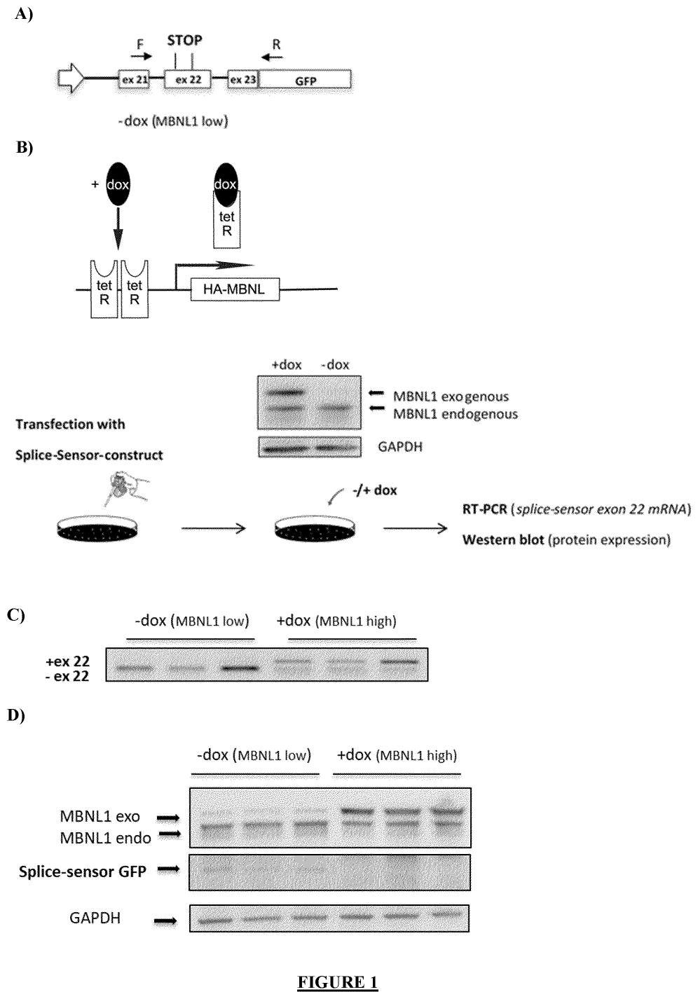

ATP2A1 exon22 is regulated by MBNL1 and contains 2 stop codons. Thus, the following splice-sensor composed by the following sequence was designed: partial 3′ of exon21-intron21-exon22-intron22-partial 5′ of exon22 of ATP2A1 gene. This splice-sensor sequence was fused to a GFP sequence within a pcDNA3 vector (FIG. 1.A). To assess whether the protein expression of the GFP transgene is regulated by MBNL1 activity, the splice-sensor-GFP construct was transfected into MBNL1-inducible HEK-293 cells (FIG. 1B). This cell line contains a tetracycline-inducible MBNL1 construct permitting to have within the same cells either high level of MBNL1 under permissive condition (+doxycycline) or low under non-permissive condition (−doxycycline). The control of MBNL1 expression in presence or absence of doxycycline is an experimental model enabling to mimic pathological state (low level of MBNL1) and physiological state (high level of MBNL1). As showed by RT-PCR in FIG. 1.C, the exon 22 of ATP2A1 is excluded in cells expressing a low level of MBNL1. In contrast this exon is included within the transgene mRNA when the level of MBNL1 is high confirming that the alternative splicing regulation of the splice-sensor ATP2A1 exon22 is MBNL1-dependent. At the protein level, the GFP was detected in cells with a low level of MBNL1 whereas no GFP expression was detected in the same cells in which the level of MBNL1 is high that leads to the inclusion of ATP2A1 exon22 containing 2 stop codons (FIG. 1.D).

### Example 2: Splice-Sensor-V5-MBNLΔ Regulation

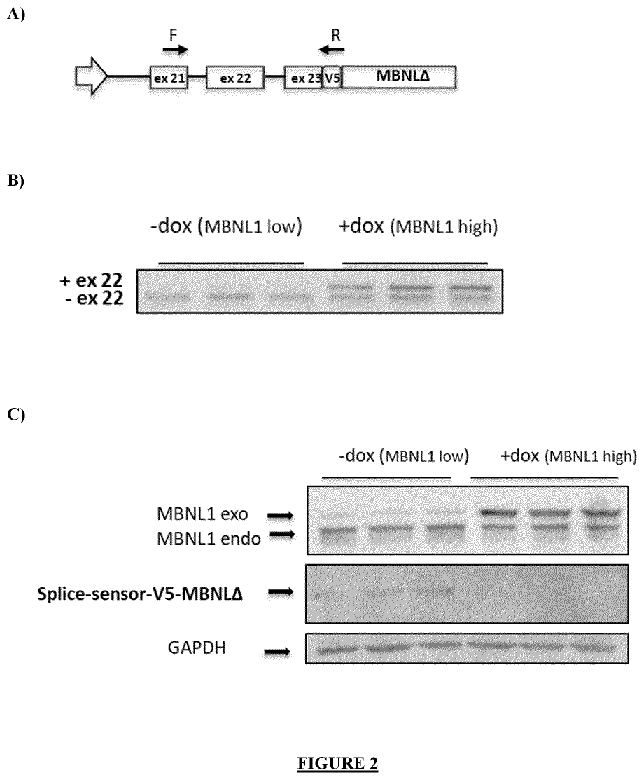

Next, we fused the same splice-sensor sequence to a modified V5-MBNLΔ construct that acts as a CUGexp-decoy to release endogenous MBNL1 from RNA foci in DM1 (as described in WO2015/158365). This transgene was transfected into MBNL1-inducible HEK-293 cells (FIG. 2.A). We confirmed that the alternative splicing regulation of the splice-sensor comprising ATP2A1 exon22 is also MBNL1-dependent (FIG. 2.B). As a consequence, the V5-MBNLΔ protein is only expressed under MBNL1 low level condition due to the inclusion of the ATP2A1 exon22 that contains stop codons (FIG. 2.C).

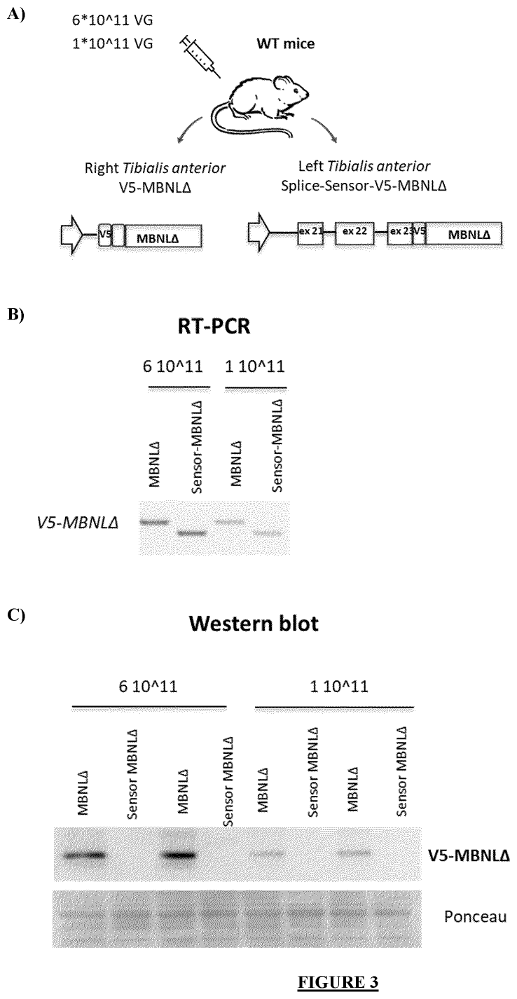

To extend the use of the splice-sensor for in vivo application, we cloned the splice-sensor-V5-MBNLΔ in a pSMD2 backbone and produced AAV9 vectors. Intramuscular injections at two different doses of either AAV9-V5-MBNLΔ or AAV9-splice-sensor-V5-MBNLΔ were performed in wild-type (WT) mice that have a normal MBNL1 activity (FIG. 3.A). RT-PCR analysis showed that V5-MBNLΔ mRNAs were expressed similarly between both constructs (FIG. 3.B). However, at the protein level, V5-MBNLΔ was detected only in muscles injected with AAV9-V5-MBNLΔ (FIG. 3.C). No expression of V5-MBNLΔ were detected in muscles injected with AAV9-splice-sensor-V5-MBNLΔ indicating that the ATP2A1 exon22 is included within the mature transcript and block transgene expression in WT context displaying normal MBNL1 activity.

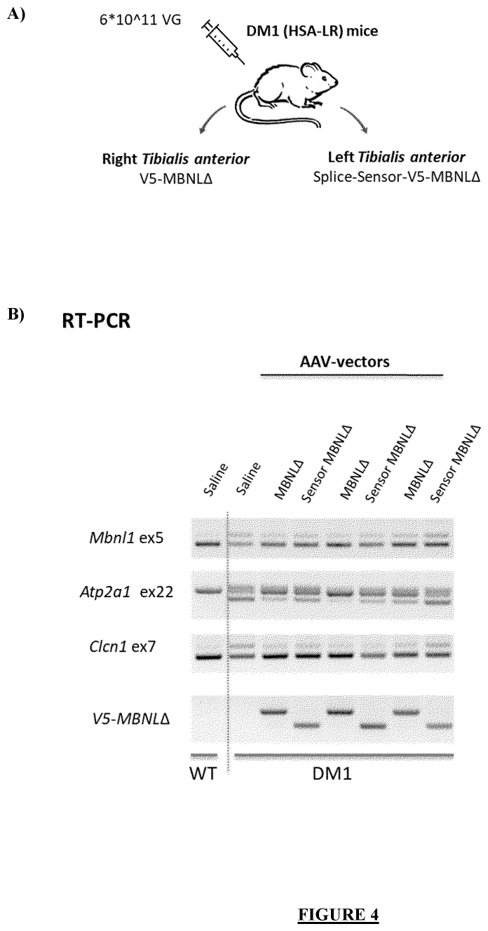

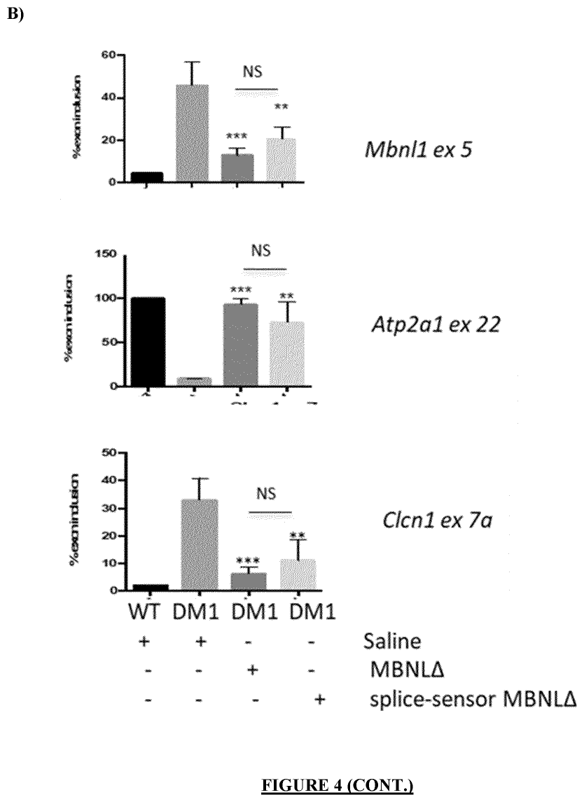

Finally, the AAV9-splice-sensor-V5-MBNLΔ was tested in DM1 mice (HSA-LR). This mouse model recapitulates molecular hallmark of the DM1 disease including expression of mutant RNA containing expanded CUG repeats that sequester MBNL1 splicing factor leading to MBNL1 functional loss and splicing defects. Intramuscular injections of either AAV9-V5-MBNLΔ or AAV9-splice-sensor-V5-MBNLΔ were performed in DM1 mice that have a reduced MBNL1 activity (FIG. 4.A). As shown in FIG. 4.B, V5-MBNLΔ mRNAs were detected in both conditions. The results show that AAV9-V5-MBNLΔ as well as AAV9-splice-sensor-V5-MBNLΔ leads to the correction of DM1 mis-splicing events in injected mice (FIG. 4.B). In fact, V5-MBNLΔ protein was detected in muscles of DM1 mice injected with both AAV-vectors (FIG. 4.C) indicating that the skipping of the ATP2A1 exon22 occurred in DM1 mice with reduced MBNL1 activity and results in the expression of V5-MBNLΔ.

In conclusion, the “splice-sensor” makes it possible to control the expression of the transgene (i.e. MBNLΔ) depending on the functional level of endogenous MBNL1. Thus the MBNLΔ protein is not produced in the WT mouse muscles while its expression in the DM1 mouse muscles is sufficient to obtain a therapeutic effect and is subjected to a self-regulation loop which regulates and limits its level of production.

### Example 3: Splice-Sensor-dCas9 Regulation

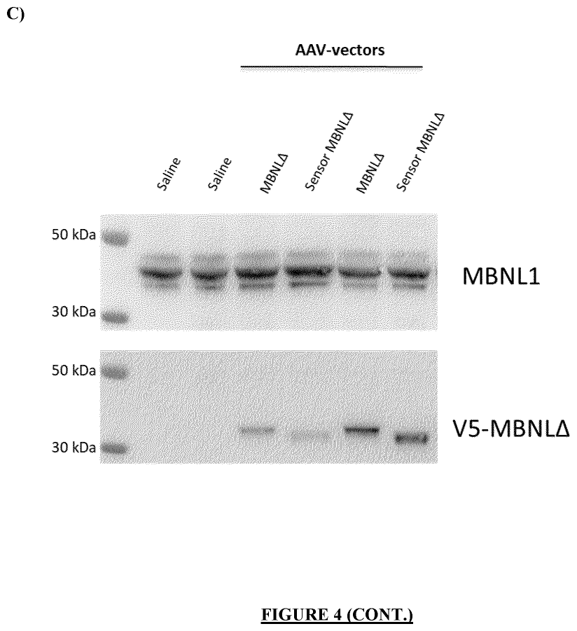

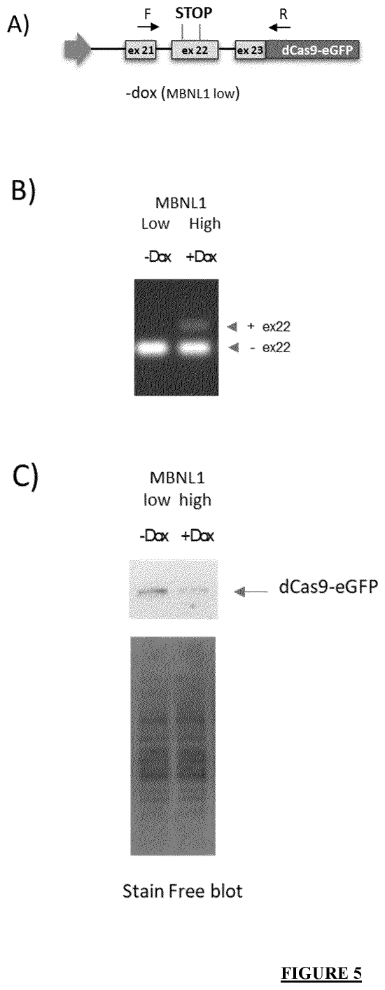

The same splice sensor construct as the one described in examples 1 and 2 (i.e. ATP2A1 splice-sensor sequence) was fused to a different transgene (i.e. dCas9-eGFP sequence) (FIG. 5.A). As a side note, Batra et al. described the therapeutic use of this dCas9 transgene (Nelles et al, Cell, 2016), fused to a PIN-endonuclease (Batra et al., Cell, 2017).

The nucleic acid molecule used in this example, which comprises the splice-sensor and the dCas9-eGFP sequence is as shown in SEQ ID NO:21.

As shown by RT-PCR in FIG. 5.B, the exon 22 of ATP2A1 is excluded in cells expressing a low level of MBNL1. In contrast this exon is included within the transgene mRNA when the level of MBNL1 is high, again confirming that the alternative splicing regulation of the splice-sensor ATP2A1 exon22 is MBNL1-dependent. At the protein level, Western blot analysis showed dCas9-eGFP expression in cells expressing low level of MBNL1 protein (−dox). However, dCas9-eGFP protein expression was reduced by 65% when the level of MBNL1 (+dox) is high, due to the inclusion of exon22.

This example demonstrates that the “splice-sensor” makes it possible to control the expression of any transgene of interest, not only MBNLΔ.

### Example 4: Embedded-Splice-Sensor-V5-MBNLΔ Regulation

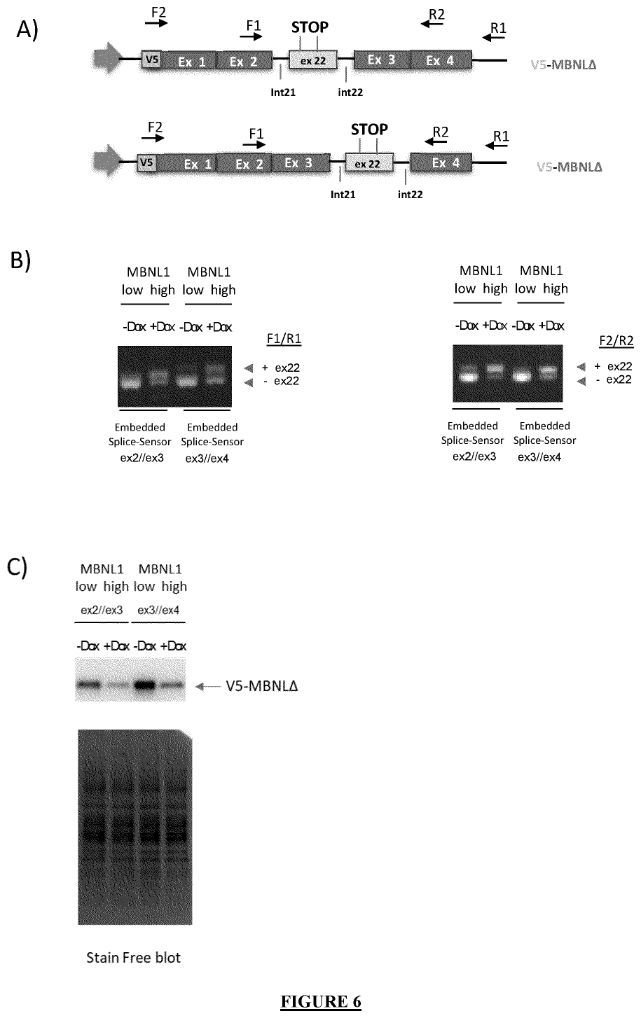

The effect of the splice sensor was then evaluated using a splice sensor “embedded” into the transgene sequence, instead of being introduced upstream of the transgene sequence. In this example, the ATP2A1 splice-sensor comprising the “intron 21-exon 22-intron 23” ATP2A1 sequence was introduced within the sequence encoding the transgene of interest, i.e. between two exons of the transgene of interest. In particular, the “Embedded-Splice-Sensor” was inserted either between MBNLΔ exon 2 and exon 3 or between MBNLΔ exon 3 and exon 4 (FIG. 6.A). An extra base (T) was inserted inside the exon 22 of the ATP2A1 embedded-splice-sensor sequence to keep the 2 STOP codons in frame.

The nucleic acid molecule used in this example, which comprises the splice-sensor inserted between MBNLΔ exon 2 and exon 3 is as shown in SEQ ID NO:23. The nucleic acid molecule used in this example, which comprises the splice-sensor inserted between MBNLΔ exon 3 and exon 4 is as shown in SEQ ID NO:24.

As shown by RT-PCR in FIG. 6.B, exon 22 of the embedded-splice-sensor that contains two codon stops, is included only when the level of MBNL1 is high (+dox). Western blot analysis showed V5-MBNLΔ protein expression in HEK293T transfected cells expressing low level of MBNL1 protein (−dox). However, when the level of MBNL1 (+dox) is high:
V5-MBNLΔ protein expression was reduced by 60% in transfected Hek293T cells, when the embedded-splice-sensor is embedded between exon 2 and exon 3;
V5-MBNLΔ protein expression was reduced by 70% in transfected Hek293T cells, when the embedded-splice-sensor is embedded between exon 3 and exon 4.

This example demonstrates the efficacy of a splice sensor inserted within a transgene sequence, for controlling the expression of the transgene.

### Example 5: 3′-Synthetic-Splice-Sensor-V5-MBNLΔ Regulation

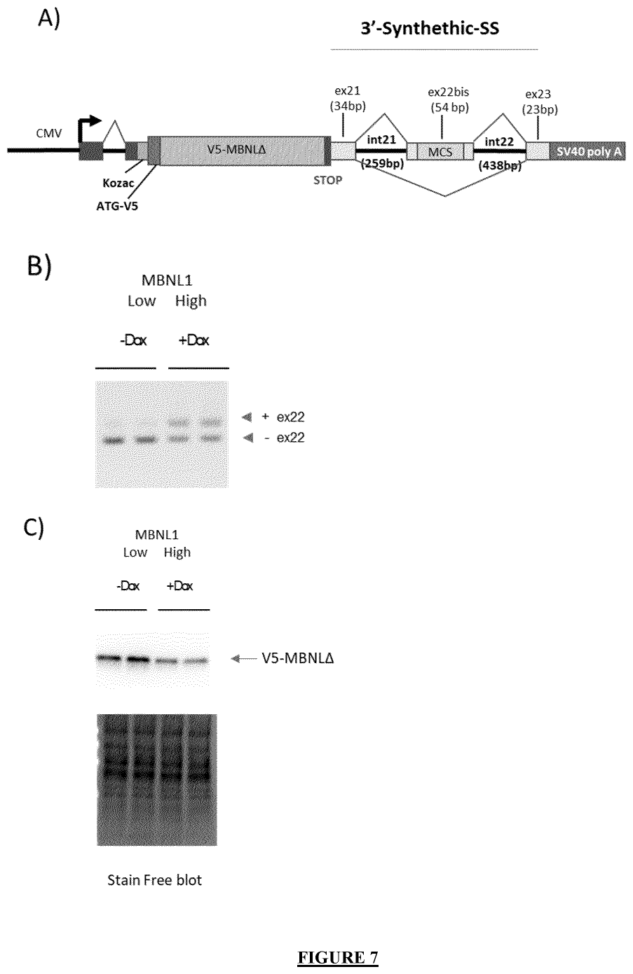

A synthetic-splice-sensor containing an additional multiple cloning site (MCS) sequence of 12 bp in exon 22 (partial 3′ of exon2l-intron2l-exon22+MCSsequence-intron22-partial 5′ of exon 23) was used in this example. Said synthetic splice sensor was introduced in 3′ of the transgene sequence, more particularly between the STOP codon and polyA of the V5-MBNLΔ sequence (FIG. 7.A).

The nucleic acid molecule used in this example, which comprises the splice-sensor inserted between the STOP codon and polyA of the V5-MBNLΔ sequence is as shown in SEQ ID NO:25.

Said sequence further comprises a Kozak sequence and an translation initiation codon (ATG) as schematized in FIG. 7.A.

As shown by RT-PCR in FIG. 7.B, exon 22 of this synthetic splice sensor is included only when the level of MBNL1 is high (+dox), again confirming that inclusion/exclusion of exon22 depends on the level of MBNL1. At the protein level, Western blot analysis showed transgene expression in cells expressing low level of MBNL1 protein (−dox). However, transgene protein expression was reduced by 50% when the level of MBNL1 (+dox) is high, due to the inclusion of exon 22.

In this example, the inhibition of transgene protein expression does not result from the occurrence of a premature STOP codon in the mature transcript of the transgene of interest, since the splice sensor was introduced after the transgene STOP codon. Thus, the inclusion of exon 22 of the splice sensor inhibits transgene protein expression through another mechanism.

In this example, the inhibition of protein expression probably results from a nonsense-mediated decay (NMD) or NMD-like mechanism, through an exon junction complex (EJC)-dependent pathway. In particular, when exon 22 of the splice sensor is included, it probably triggers an exon junction complex (a large multi-protein assembly) located downstream of the stop codon of the transgene. However, if an EJC is present downstream of the stop codon, said stop codon will be considered as an aberrant premature stop codon (PSC) that will inhibit proper release of the mRNA by the ribosomes, resulting in reduced translation probably due to the degradation of the mRNA transcript by a NMD or NMD-like mechanism.

Thus, this example demonstrates that the expression of a transgene of interest can be controlled with a synthetic splice sensor which may be inserted after the stop codon of the transgene. In addition, it may be concluded that the splice sensor may control the protein expression via different mechanisms, such as via the inclusion of a premature STOP codon within the mature transcript or via the degradation of mRNA transcript by a NMD or NMD-like mechanism.

### Example 6: 3′-Murine-Splice-Sensor-V5-MBNLΔ Regulation

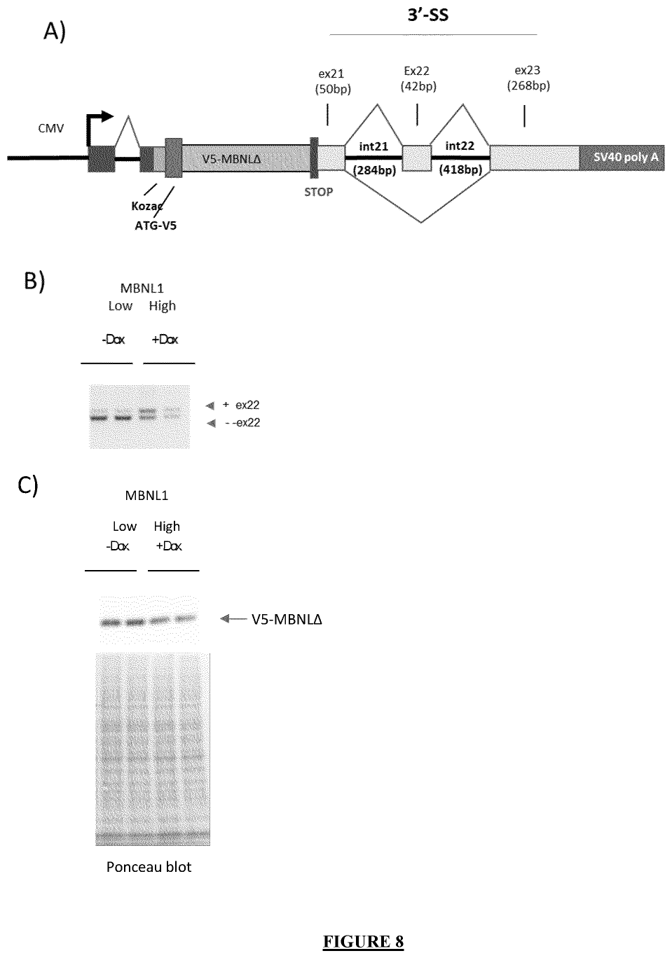

The splice sensor used in this example is derived from the murine ATP2A1 sequence and comprises the entire exon 23 (ex21partial-int21-ex22-int22-ex23). Said murine splice sensor was introduced in 3′ of the transgene sequence, more particularly between the STOP codon and polyA of the V5-MBNLΔ sequence (FIG. 8.A).

The nucleic acid molecule used in this example, which comprises the murine splice-sensor inserted between the STOP codon and polyA of the V5-MBNLΔ sequence is as shown in SEQ ID NO:26. Said sequence further comprises a Kozak sequence and an translation initiation codon (ATG) as schematized in FIG. 8.A.

As shown by RT-PCR in FIG. 8.B, exon 22 of this murine splice sensor is included only when the level of MBNL1 is high (+dox), again confirming that inclusion/exclusion of exon22 depends on the level of MBNL1. At the protein level, Western blot analysis showed transgene expression in cells expressing low level of MBNL1 protein (−dox). However, transgene protein expression was reduced by 40% when the level of MBNL1 (+dox) is high, due to the inclusion of exon 22.

This example demonstrates that a different splice sensor, in particular deriving from a murine sequence, may be used for controlling the expression of a transgene of interest.

### CONCLUSION

The above-exemplified splice-sensor is thus of particular interest for gene therapy of DM1, since it ensures the expression of the therapeutic transgene only in tissues of cells that needed it, i.e. in tissues or cells wherein MBNL1 activity is reduced. The examples demonstrate that the splice sensor may be used for controlling the expression of any transgene of interest (such as MBNLΔ or a therapeutic endonuclease such as Cas9). In addition, the efficacy of the splice sensor was demonstrated using several splice-sensor sequences (i.e. natural human ATP2A1 sequence, artificial sequence or murine ATP2A1 sequence). Of course, the splice-sensor may comprise any other intron-exon-intron sequence for which the inclusion of said exon in the mature transcript is triggered by MBNL1. In addition, the above examples demonstrate that the exon of the splice-sensor is able to inhibit the protein translation of the mRNA transgene, through different mechanisms. In particular, the inclusion of the exon in the mature transcript may lead to the occurrence of a premature STOP codon in said mature transcript. In addition, the inclusion of the exon in the mature transcript may inhibit the protein translation by a NMD or NMD-like mechanism. As shown in the examples, the splice sensor may be inserted upstream the transgene sequence of interest or within the transgene sequence of interest (between 2 exonic sequences or between the STOP codon and polyA).

These examples using a splice-sensor targeted by MBNL1 provide a successful proof of concept. Of course, this system may be adapted for regulating the expression of any transgene of interest into a cell or a host organism, depending on the activity of any protein regulating alternative splicing (other than MBNL1 protein).

### REFERENCES

Batra R, Nelles D A, Pirie E, et al. Elimination of Toxic Microsatellite Repeat Expansion RNA by RNA-Targeting Cas9. Cell. 2017; 170(5):899-912.e10.
Brook, J. D., M. E. McCurrach, et al. (1992). “Molecular basis of myotonic dystrophy: expansion of a trinucleotide (CTG) repeat at the 3′ end of a transcript encoding a protein kinase family member.” Cell 69(2): 385.
Fardaei, M., K. Larkin, et al. (2001). “In vivo co-localisation of MBNL protein with DMPK expanded-repeat transcripts.” Nucleic Acids Res 29(13): 2766-71.
Kanadia, R. N., J. Shin, et al. (2006). “Reversal of RNA missplicing and myotonia after muscleblind overexpression in a mouse poly(CUG) model for myotonic dystrophy.” Proc Natl Acad Sci USA 103(31): 11748-53.
Miller, J. W., C. R. Urbinati, et al. (2000). “Recruitment of human muscleblind proteins to (CUG)(n) expansions associated with myotonic dystrophy.” EMBO J 19(17): 4439-48.
Nelles D A, Fang M Y, O'Connell M R, et al. Programmable RNA Tracking in Live Cells with CRISPR/Cas9. Cell. 2016; 165(2):488-496.
Tran, H., N. Gourrier, et al. (2011). “Analysis of exonic regions involved in nuclear localization, splicing activity, and dimerization of Muscleblind-like-1 isoforms.” J Biol Chem 286(18): 16435-46.

1-22. (canceled)

23. A chimeric nucleic acid molecule comprising:
a first nucleic acid sequence, the primary transcript of which comprises an exon, said primary transcript being subject to splicing by a protein regulating alternative splicing; and
a second nucleic acid sequence comprising a transgene of interest encoding a product of interest;
wherein the protein regulating alternative splicing enhances the inclusion of said exon in the mature transcript of the chimeric nucleic acid molecule, and
wherein said exon is designed to inhibit expression of a functional product of interest, when included into the mature transcript.

24. The nucleic acid molecule of claim 23, wherein the first nucleic acid sequence is located:
upstream of the second nucleic acid sequence; or
within the second nucleic acid sequence or between two exons of said transgene of interest; or
in the 3′UTR region of the second nucleic acid sequence or between the termination codon and the polyA sequence.

25. The nucleic acid molecule of claim 23, wherein the transgene of interest encodes the protein regulating alternative splicing, or a variant thereof.

26. The nucleic acid molecule of claim 23, wherein the protein regulating alternative splicing is a MBNL protein, MBNL1 protein, MBNL2 protein, MBNL3 protein or a variant of said proteins.

27. The nucleic acid molecule of claim 23, wherein the transgene of interest encodes a modified MBNL protein having an YGCY binding property and having a reduced splicing activity as compared to wild-type MBNL protein.

28. The nucleic acid molecule of claim 27, wherein said modified MBNL protein binds CUG repeats.

29. The nucleic acid molecule of claim 27, wherein said modified MBNL protein is lacking the C-terminal domain of the wild-type MBNL protein.

30. The nucleic acid molecule of claim 27, wherein said modified MBNL protein is derived from the MBNL1 protein and is lacking the amino acids corresponding to the encoding exons 5 to 10 of the MBNL1 mRNA.

31. The nucleic acid molecule of claim 27, wherein said modified MBNL protein comprises SEQ ID NO: 5, or a functional YGCY-binding variant thereof.

32. The nucleic acid molecule of claim 27, wherein said modified MBNL protein has a splicing activity reduced by at least 50% as compared to the wild-type MBNL protein.

33. The nucleic acid molecule of claim 23, wherein the inclusion of the exon in the mature transcript of the chimeric nucleic acid molecule leads to the occurrence of a premature STOP codon in said mature transcript.

34. The nucleic acid molecule of claim 23, wherein the exon includes a STOP codon.

35. The nucleic acid molecule of claim 23, wherein the exon of the first sequence is flanked by two introns.

36. The nucleic acid molecule of claim 23, wherein the exon is exon 22 of the ATP2A1 gene, exon 70 of the RYR1 gene, exon 8 of the CAPZB gene, exon 11 of the INSR gene, exon 13 of the CAM2B gene, exon 17 of the ITGB gene, exon 11 of the BIN1 gene, exon 2 or 3 of the TAU gene or exon 78 of the DMD gene.

37. The nucleic acid molecule of claim 35, wherein exon 22 is flanked by intron 21 and intron 22 of the ATP2A1 gene.

38. An expression cassette comprising the nucleic acid molecule of claim 23, operably linked to regulatory sequences.

39. A vector comprising the nucleic acid molecule of claim 23 or an expression cassette comprising said nucleic acid operably linked to regulatory sequences.

40. The vector of claim 39, wherein said vector is a plasmid or a viral vector.

41. An isolated cell transformed with the nucleic acid molecule of claim 23, an expression cassette comprising said nucleic acid operably linked to regulatory sequences, or a vector comprising said nucleic acid molecule or expression cassette.

42. A method of treating a disease or disorder linked to a dysfunction of the protein regulating alternative splicing comprising administering to a subject having said disease or disorder the nucleic acid of claim 23, an expression cassette comprising said nucleic acid operably linked to regulatory sequences, a vector comprising said nucleic acid molecule or expression cassette, or a cell transformed with said nucleic acid molecule, an expression cassette comprising said nucleic acid operably linked to regulatory sequences, or a vector comprising said nucleic acid molecule or expression cassette.

43. A method of treating a disease or disorder linked to a sequestration of MBNL, myotonic dystrophy, Myotonic dystrophy type 1, or Myotonic dystrophy type 2 comprising administering to a subject in need of treatment the nucleic acid of claim 23, an expression cassette comprising said nucleic acid operably linked to regulatory sequences, a vector comprising said nucleic acid molecule or expression cassette, or a cell transformed with said nucleic acid molecule, an expression cassette comprising said nucleic acid operably linked to regulatory sequences, or a vector comprising said nucleic acid molecule or expression cassette.

44. A method for controlling the expression of the transgene of interest, depending on the endogenous level of the protein regulating alternative splicing, comprising the expression of the nucleic acid of claim 23, an expression cassette comprising said nucleic acid operably linked to regulatory sequences, a vector comprising said nucleic acid molecule or expression cassette in a cell.


SEQUENCE LISTING (31 sequences)

SEQ ID NO: 1 - protein, 400 aa, Homo sapiens
MAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAMTQSAVKSLKRPLEATFDLGIPQAVLPPLPKRPALEKTNGATAVFNTGIFQYQQALANMQLQQHTAFLPPGSILCMTPATSVVPMVHGATPATVSAATTSATSVPFAATATANQIPIISAEHLTSHKYVTQM

SEQ ID NO: 2 - protein, 391 aa, Homo sapiens
MALNVAPVRDTKWLTLEVCRQFQRGTCSRSDEECKFAHPPKSCQVENGRVIACFDSLKGRCSRENCKYLHPPTHLKTQLEINGRNNLIQQKTAAAMLAQQMQFMFPGTPLHPVPTFPVGPAIGTNTAISFAPYLAPVTPGVGLVPTEILPTTPVIVPGSPPVTVPGSTATQKLLRTDKLEVCREFQRGNCARGETDCRFAHPADSTMIDTSDNTVTVCMDYIKGRCMREKCKYFHPPAHLQAKIKAAQHQANQAAVAAQAAAAAATVMTQSTAKAMKRPLEATVDLAFPPGALHPLPKRQALEKSNGTSAVFNPSVLHYQQALTSAQLQQHAAFIPTGSVLCMTPATSIDNSEIISRNGMECQESALRITKHCYCTYYPVSSSIELPQTAC

SEQ ID NO: 3 - protein, 354 aa, Homo sapiens
MTAVNVALIRDTKWLTLEVCREFQRGTCSRADADCKFAHPPRVCHVENGRVVACFDSLKGRCTRENCKYLHPPPHLKTQLEINGRNNLIQQKTAAAMFAQQMQLMLQNAQMSSLGSFPMTPSIPANPPMAFNPYIPHPGMGLVPAELVPNTPVLIPGNPPLAMPGAVGPKLMRSDKLEVCREFQRGNCTRGENDCRYAHPTDASMIEASDNTVTICMDYIKGRCSREKCKYFHPPAHLQARLKAAHHQMNHSAASAMALQPGTLQLIPKRSALEKPNGATPVFNPTVFHCQQALTNLQLPQPAFIPAGPILCMAPASNIVPMMHGATPTTVSAATTPATSVPFAAPTTGNQLKF

SEQ ID NO: 4 - protein, 131 aa, Homo sapiens
TQSAVKSLKRPLEATFDLGIPQAVLPPLPKRPALEKTNGATAVFNTGIFQYQQALANMQLQQHTAFLPPGSILCMTPATSVVPMVHGATPATVSAATTSATSVPFAATATANQIPIISAEHLTSHKYVTQM

SEQ ID NO: 5 - protein, 269 aa, Artificial
  MBNLdelta
MAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAM

SEQ ID NO: 6 - protein, 201 aa, Artificial
  MBNLdeltaCT (without exon 3)
MAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVVCREYQRGNCNRGENDCRFAHPADSTMIDTNDNTVTVCMDYIKGRCSREKCKYFHPPAHLQAKIKAAQYQVNQAAAAQAAATAAAM

SEQ ID NO: 7 - protein, 268 aa, Artificial
  MBNL2deltaCT3
MALNVAPVRDTKWLTLEVCRQFQRGTCSRSDEECKFAHPPKSCQVENGRVIACFDSLKGRCSRENCKYLHPPTHLKTQLEINGRNNLIQQKTAAAMLAQQMQFMFPGTPLHPVPTFPVGPAIGTNTAISFAPYLAPVTPGVGLVPTEILPTTPVIVPGSPPVTVPGSTATQKLLRTDKLEVCREFQRGNCARGETDCRFAHPADSTMIDTSDNTVTVCMDYIKGRCMREKCKYFHPPAHLQAKIKAAQHQANQAAVAAQAAAAAATVM

SEQ ID NO: 8 - protein, 257 aa, Artificial
  MBNL3DeltaCT3
MTAVNVALIRDTKWLTLEVCREFQRGTCSRADADCKFAHPPRVCHVENGRVVACFDSLKGRCTRENCKYLHPPPHLKTQLEINGRNNLIQQKTAAAMFAQQMQLMLQNAQMSSLGSFPMTPSIPANPPMAFNPYIPHPGMGLVPAELVPNTPVLIPGNPPLAMPGAVGPKLMRSDKLEVCREFQRGNCTRGENDCRYAHPTDASMIEASDNTVTICMDYIKGRCSREKCKYFHPPAHLQARLKAAHHQMNHSAASAM

SEQ ID NO: 9 - DNA, 796 bp, Artificial
  splice sensor
atgctcaagttcgttgctcggaactacctagagggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggataactgttccccctcctccatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagatccagaagatgaaagaaggaag

SEQ ID NO: 10 - protein, 7 aa, Artificial
  NLS
PKKKRKV

SEQ ID NO: 11 - protein, 10 aa, Artificial
  NES
LPPLERLTLD

SEQ ID NO: 12 - protein, 14 aa, Artificial
  V5 tag
GKPIPNPLLGLDST

SEQ ID NO: 13 - protein, 283 aa, Artificial
  V5+MBNLdelta
MGKPIPNPLLGLDSTAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAM

SEQ ID NO: 14 - DNA, 1642 bp, Artificial
  splice-sensor ATP2A1 ex21-intron-exon22-intron- ex23-V5-MBNLdelta
atgctcaagttcgttgctcggaactacctagagggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggataactgttccccctcctccatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagatccagaagatgaaagaaggaagggtaagcctatccctaaccctctcctcggtctcgattctacggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatg

SEQ ID NO: 15 - protein, 301 aa, Artificial
  Splice-Sensor ATP2A1 ex21-ex23-v5- Cter MBNLdelta (exon 22 skipped)
MLKFVARNYLEDPEDERRKGKPIPNPLLGLDSTAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAM

SEQ ID NO: 16 - protein, 12 aa, Artificial
  ex21-ex22-ex23-v5-MBNLdelta (exon 22 included, early stop)
MLKFVARNYLEG

SEQ ID NO: 17 - DNA, 807 bp, Artificial
  MBNLdelta
atggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatg

SEQ ID NO: 18 - DNA, 849 bp, Artificial
  V5+MBNLdelta
atgggtaagcctatccctaaccctctcctcggtctcgattctacggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatg

SEQ ID NO: 19 - DNA, 1600 bp, Artificial
  Splice-Sensor ATP2A1 ex21-ex23-MBNLdelta
atgctcaagttcgttgctcggaactacctagagggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggataactgttccccctcctccatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagatccagaagatgaaagaaggaaggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatg

SEQ ID NO: 20 - protein, 287 aa, Artificial
  Splice-Sensor ATP2A1 ex21-ex23-MBNLdelta (exon 22 skipped)
MLKFVARNYLEDPEDERRKAVSVTPIRDTKWLTLEVCREFQRGTCSRPDTECKFAHPSKSCQVENGRVIACFDSLKGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAM

SEQ ID NO: 21 - DNA, 5716 bp, artificial
  Splice-Sensor-dCas9-HA-NLSx2-eGFP
atgctcaagttcgttgctcggaactacctagagggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggataactgttccccctcctccatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagatccagaagatgaaagaaggaagggtaccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 22 - protein, 1659 aa, artificial
  Splice-Sensor-dCas9-HA-NLSx2-eGFP (ex 22 skipped)
MLKFVARNYLEDPEDERRKGTDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - DNA, 1589 bp, artificial
  v5-MBNLdelta1(ex1/2)-Embedded-Splice-Sensor- MBNLdelta1(ex3/4)
atgggtaagcctatccctaaccctctcctcggtctcgattctacggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggattaactgttccccctcctccatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatg

SEQ ID NO: 24 - DNA, 1589 bp, artificial
  v5-MBNLdelta1(ex1/2/3)-Embedded-Splice-Sensor- MBNLdelta1(ex4)
atgggtaagcctatccctaaccctctcctcggtctcgattctacggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggattaactgttccccctcctccatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatg

SEQ ID NO: 25 - DNA, 1673 bp, artificial
  V5-MBNLdelta-3′-Synthetic-Splice-Sensor
taccggtcgccaccatgggtaagcctatccctaaccctctcctcggtctcgattctacggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgtaatgctcaagttcgttgctcggaactacctagagggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggataactgttccccctcctccgatatctctagaatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagatccagaagatgaaagaaggaag

SEQ ID NO: 26 - DNA, 1929 bp, artificial
  V5-MBNLdelta-3′-Splice-Sensor
taccggtcgccaccatgggtaagcctatccctaaccctctcctcggtctcgattctacggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgtaacatcgggctggatgagcttctcaagttcattgctcggaactatctggagggtaagggacatccctttttccttgtacccagtcctgagcagagcccctgccctctgcgctgcgaacacgcgccataagtcatctatccccctcttcctttgtaggtaggtaggtaagttctttttagccctggtgggaccagtgtcccccagggagggcaacccaagccctgtccccactccagctatgactggtgggattttctcccaacctgctcagccccctggctccgagggcctccttccctcttgataacggcctctctctgtcctctctggccataggataaccacccccctcctccatgtctttgaaccgtgtcacaggtatttccctacccgccctgccccagctgctgtaccctgccgctcgcccaccaccagcccatgcgcacccctgcggtcacttgcgcgcgctcgcagctctccctggagccactgctgctgttgccactgcccgcttccacatgagggccatgccgttactgtgggcactggcagctgtgaggctgtgtcctggccgtccagtctgctgggctgggatgcgctgcgctgcgtcgcgctggctgtctctgctgctggggctgcggtggtggctggggggcgggttggggtctggcacatcacaggtgagtagggaaaggggcccttaggcccctccaggtgagtgtgcaggagatgcgcaaggagatacagctctccctcctgtgcccgcctgctccatcccctgcctttcttcttccagatccagaagacgaaagaaggaagtaaccagcccttgtgccctgtgttccccaccccaatagccatgcatcttcagccaaagctgaggcactgacccttcttccccctcaattctgtttataaataaagtcctccaccctcgtccccttcagcaacccgctctccctccgaaccttgtaaatccccttcccgaactcacggggcttgcaggcacaaggcaactgactagctgattgcttatttattgaaaataaatgacagaaaagtctg

SEQ ID NO: 27 - protein, 1638 aa, artificial
  dCas9-eGFP protein
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 28 - DNA, 4914 bp, artificial
  dCas9-eGFP
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 29 - DNA, 740 bp, artificial
  splice sensor  int21-ex22(+1bp)-int22
gtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggattaactgttccccctcctccatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccag

SEQ ID NO: 30 - DNA, 807 bp, artificial
  splice sensor  ex21partial-in21-ex22/MCS(54bp)- int22-ex23partial
tgctcaagttcgttgctcggaactacctagagggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggataactgttccccctcctccgatatctctagaatctctgagcccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagatccagaagatgaaagaaggaag

SEQ ID NO: 31 - DNA, 1063 bp, artificial
  splice sensor  ex21partial-in21-ex22-int22- ex23. (murine Atp2a1 sequence)
catcgggctggatgagcttctcaagttcattgctcggaactatctggagggtaagggacatccctttttccttgtacccagtcctgagcagagcccctgccctctgcgctgcgaacacgcgccataagtcatctatccccctcttcctttgtaggtaggtaggtaagttctttttagccctggtgggaccagtgtcccccagggagggcaacccaagccctgtccccactccagctatgactggtgggattttctcccaacctgctcagccccctggctccgagggcctccttccctcttgataacggcctctctctgtcctctctggccataggataaccacccccctcctccatgtctttgaaccgtgtcacaggtatttccctacccgccctgccccagctgctgtaccctgccgctcgcccaccaccagcccatgcgcacccctgcggtcacttgcgcgcgctcgcagctctccctggagccactgctgctgttgccactgcccgcttccacatgagggccatgccgttactgtgggcactggcagctgtgaggctgtgtcctggccgtccagtctgctgggctgggatgcgctgcgctgcgtcgcgctggctgtctctgctgctggggctgcggtggtggctggggggcgggttggggtctggcacatcacaggtgagtagggaaaggggcccttaggcccctccaggtgagtgtgcaggagatgcgcaaggagatacagctctccctcctgtgcccgcctgctccatcccctgcctttcttcttccagatccagaagacgaaagaaggaagtaaccagcccttgtgccctgtgttccccaccccaatagccatgcatcttcagccaaagctgaggcactgacccttcttccccctcaattctgtttataaataaagtcctccaccctcgtccccttcagcaacccgctctccctccgaaccttgtaaatccccttcccgaactcacggggcttgcaggcacaaggcaactgactagctgattgcttatttattgaaaataaatgacagaaaagtctg